(19) United States
(12) Patent Application Publication
(10) Pub. No.: US 2017/0156801 A1
(43) Pub. Date: Jun. 8, 2017

(54) SYSTEMS AND METHODS FOR TREATING TISSUE WITH RADIOFREQUENCY ENERGY

(71) Applicant: Mederi Therapeutics, Inc., Norwalk, CT (US)

(72) Inventor: OLEG SHIKHMAN, Trumbull, CT (US)

(73) Assignee: Mederi Therapeutics, Inc., Norwalk, CT (US)

(21) Appl. No.: 15/436,827

(22) Filed: Feb. 19, 2017

Related U.S. Application Data
(63) Continuation-in-part of application No. 15/068,539, filed on Mar. 12, 2016.
Application No. 15/068,539 is a continuation of application No. 13/646,681, filed on Oct. 6, 2012, now Pat. No. 9,292,152.
Application No. 13/646,681 is a continuation of application No. 12/924,155, filed on Sep. 22, 2010, now abandoned.
(60) Provisional application No. 62/313,710, filed on Mar. 26, 2016.
(60) Provisional application No. 61/277,260, filed on Sep. 22, 2009.

(51) Int. Cl. A61B 34/00; G06T 11/60; A61B 34/20; A61B 18/14; A61B 18/12

(52) U.S. Cl. CPC A61B 34/25; A61B 18/1485; A61B 18/1206; A61B 34/20; G06T 11/60; A61B 2034/254; A61B 2018/00553

(57) ABSTRACT

A system for controlling a treatment device includes a system for visually tracking different axially spaced tissue levels and treatment regions within each axially spaced tissue level for application of radiofrequency energy by electrodes of a device to a body region of a patient to perform a surgical procedure, the system comprising a graphic display configured to display a plurality of tissue level indicators, each tissue level indicator corresponding to one of the different axially spaced tissue levels, and a numeric indicator associated with each tissue level indicator configured to indicate a distance from a fixed reference to a designated region within the patient, the numeric indicator automatically changing in response to axial repositioning of the electrodes of the device within the patient.

### RELATED APPLICATIONS

This application claims priority from provisional application No. 62/313,710, filed Mar. 26, 2016, and is a continuation in part of application Ser. No. 14/953,214, filed Nov. 27, 2015, which is a continuation of U.S. application Ser. No. 13/646,683, filed Oct. 6, 2012, which is a continuation of U.S. application Ser. No. 12/924,155, filed Sep. 22, 2010 which claims the benefit of provisional application Ser. No. 61/277,260, filed Sep. 22, 2009 and this application is also a continuation in part of application Ser. No. 13/867,042, filed Apr. 20, 2013, which claims the benefit of provisional application Ser. No. 61/664,960, filed Jun. 27, 2012 and is a continuation-in-part of application Ser. No. 12/924,155, filed Sep. 22, 2010 which claims the benefit of provisional application Ser. No. 61/277,260 filed 22 Sep. 2009. The entire contents of each of these applications are incorporated herein by reference.

### FIELD OF THE INVENTION

This invention is directed to systems and methods for treating interior tissue regions of the body. More specifically, the invention is directed to systems and methods for treating dysfunction in body sphincters and adjoining tissue.

### BACKGROUND OF THE INVENTION

The gastrointestinal (GI) tract, also called the alimentary canal, is a long tube through which food is taken into the body and digested. The alimentary canal begins at the mouth, and includes the pharynx, esophagus, stomach, small and large intestines, and rectum. In human beings, this passage is about 30 feet (9 meters) long.

Small, ring-like muscles, called sphincters, surround portions of the alimentary canal. In a healthy person, these muscles contract or tighten in a coordinated fashion during eating and the ensuing digestive process, to temporarily close off one region of the alimentary canal from another region of the alimentary canal.

For example, a muscular ring called the lower esophageal sphincter (or LES) surrounds the opening between the esophagus and the stomach. Normally, the lower esophageal sphincter maintains a high-pressure zone between fifteen and thirty mm Hg above intragastric pressures inside the stomach.

In the rectum, two muscular rings, called the internal and external sphincter muscles, normally keep fecal material from leaving the anal canal. The external sphincter muscle is a voluntary muscle, and the internal sphincter muscle is an involuntary muscle. Together, by voluntary and involuntary action, these muscles normally contract to keep fecal material in the anal canal.

Dysfunction of a sphincter in the body can lead to internal damage or disease, discomfort, or otherwise adversely affect the quality of life. For example, if the lower esophageal sphincter fails to function properly, stomach acid may rise back into the esophagus. Heartburn or other disease symptoms, including damage to the esophagus, can occur. Gastrointestinal reflux disease (GERD) is a common disorder, characterized by spontaneous relaxation of the lower esophageal sphincter.

Damage to the external or internal sphincter muscles in the rectum can cause these sphincters to dysfunction or otherwise lose their tone, such that they can no longer sustain the essential fecal holding action. Fecal incontinence results, as fecal material can descend through the anal canal without warning, stimulating the sudden urge to defecate. The physical effects of fecal incontinence (i.e., the loss of normal control of the bowels and gas, liquid, and solid stool leakage from the rectum at unexpected times) can also cause embarrassment, shame, and a loss of confidence, and can further lead to mental depression.

In treating such disorders, lesions are formed at several axially spaced lesion levels of tissue. It would be advantageous to facilitate tracking of the treatment, i.e., formation of lesions, at each tissue level and facilitate positioning of the device at the appropriate site for treatment. Currently, in order to locate the Z-line, an endoscope with markings is inserted through a bite block to the target site. The distance is measured from the Z-line to the bite block. The clinician then needs to record or remember such distance. A guidewire is inserted through the endoscope, the endoscope is removed and the treatment device (catheter) is inserted over the guidewire without visualization to a first treatment position which is preferably 1 cm above the Z-line, based on the clinician's previous memorized or recorded measurement. Similarly, for the dentate line, after visually observing the marker on the device inserted through the anal canal to mark the distance from the anal verge to the dentate line, the clinician needs to remember or record the distance. If the clinician forgets to properly record the distance or does not remember the measurement accurately, the treatment device will be positioned at the wrong location and treatment will not begin at the desired distance with respect to the Z-line or dentate line. The problem becomes compounded since the treatment device is advanced at predetermined increments for treatment at various tissue levels. It would be advantageous to provide a system that minimizes such problems by better assisting the clinician with accurate placement of the treatment device.

### SUMMARY OF THE INVENTION

One aspect of the invention provides systems and methods for treating body tissue that comprise generating a graphical display for visually prompting a user in a step-wise fashion to use a treatment device to perform a process of forming a pattern of lesions in a body region comprising a plurality of axially spaced lesion levels, each lesion level comprising a plurality of circumferential spaced lesions. The systems and methods include registering the formation of lesions as they are generated in real time, both within and between each circumferentially spaced level, whereby the graphical display displays for the user a visual record of the progress of the process from start to finish and guides the user so that individual lesions desired within a given level are all formed, and that a given level of lesions is not skipped.

In one embodiment, the systems and methods include generating at each lesion level a first stylized graphical image with a number identification of its level, and generating a second stylized graphical image, different from the first stylized graphical image, generated when the formation of lesions at a given level is indicated and further showing the number of lesions to be formed at that level. The systems and methods include changing the second graphical image to a third graphical image, different than the first or second images, including added indicia to reflect the formation of lesions in real time. The systems and methods can further include generating, upon forming the desired lesion pattern on the respective lesion level, a fourth graphical image, different than the first, second, and third graphical images, comprising an indicator to indicate that all desired lesions have been formed at the level. The systems and methods can further include generating a marker that directs the user to the next lesion level to be treated and that is updated as successive lesion levels are treated.

In accordance with another aspect, the present invention provides a system for visually tracking different axially spaced tissue levels (levels of tissue) and treatment regions within each axially spaced tissue level for application of radiofrequency energy by electrodes of a device to a body region of a patient to perform a surgical procedure. The system comprises a graphic display configured to display a plurality of tissue level indicators, each tissue level indicator corresponding to one of the different axially spaced tissue levels, and a numeric indicator associated with each tissue level indicator configured to indicate a distance from a fixed reference to a designated region within the patient. The numeric indicator automatically changes in response to axial repositioning of the electrodes of the device within the patient.

In some embodiments, the numeric indicator of a first selected tissue level of the axially spaced tissue levels is configured to disappear when a second tissue level of the axially spaced tissue levels is selected and a different numeric indicator appears.

In some embodiments, the system includes a controller, and a measurement is made from the fixed reference outside the patient to a Z-line within a gastro-intestinal tract of the patient and inputted to the controller. In some embodiments, a first treatment of the surgical procedure is a first distance from the Z-line and a subsequent second treatment of the surgical procedure is a second closer distance to the Z-line. In other embodiments, the system includes a controller, and a measurement is made from the fixed reference to a dentate line within the patient and inputted to the controller. In some embodiments, a first treatment of the surgical procedure is adjacent the dentate line and a subsequent second treatment of the surgical procedure is further from the dentate line.

In some embodiments, the tissue level indicator comprises a geometric shape and the numeric indicator is positioned within the geometric shape.

In some embodiments, after treatment of each of the treatment regions within the tissue level of the axially spaced tissue levels, a region of the tissue level indicator is configured to be visually indicated as treated and regions of the tissue level not treated are not so indicated. In some embodiments, a first tissue level indicator of the plurality of tissue level indicators is configured to provide an indication of a number of lesions formed and a number of lesions not formed at a first tissue level of the axially spaced tissue levels, and the first tissue level indicator remains viewable at the same time as a second tissue level of the axially spaced tissue levels is treated and a number of lesions formed and a number of lesions not formed are configured to be displayed on the second tissue level indicator of the plurality of tissue level indicators.

In some embodiments, each tissue level indicator of the plurality of tissue level indicators is configured to occupy a first position prior to treatment at a corresponding tissue level of the axially spaced tissue levels and a second different position during treatment at the corresponding tissue level.

In some embodiments, the graphic display is configured to display an electrode array icon corresponding to an electrode array of the device connected to the system. In some embodiments, the graphic display is further configured to display an indication of at least one measured parameter of the electrodes of the electrosurgical device connected to the system.

In accordance with another aspect of the present invention, a system is provided for visually tracking distances from a fixed reference in a surgical procedure for forming a plurality of tissue lesions within a patient within a series of levels of tissue, the series of levels of tissue being axially spaced and the fixed reference determined prior to the start of application of electrosurgical energy to a first level of tissue of the series of levels of tissue. The system comprises a controller to receive an input from a clinician corresponding to a measured distance from the fixed reference to a designated region in the patient and a graphic display configured to display a series of distances computed in response to the input of the measured distance to provide a visual indication of distances from the fixed reference to the designated region for creation of lesions at different levels of tissue during the surgical procedure.

In some embodiments, only the distance of a selected level of tissue of the series of levels of tissue is configured to be displayed while the distances of other non-selected levels of tissue of the series of levels of tissue are not displayed. In some embodiments, the measured distance is displayed simultaneously with a distance of a selected level of tissue from the fixed reference.

In some embodiments, when treatment of a first level of tissue of the series of levels of tissue is completed and a second level of tissue of the series of levels of tissue is selected, a first indicator corresponding to the first level of tissue and a second indicator corresponding to the second level of tissue are configured to move to different positions.

In some embodiments the designated region in the patient is one of a Z-line or dentate line.

In some embodiments, an adjustment button is provided to adjust a preset value to match the measured distance. In some embodiments, the adjustment button is disabled during application of electrosurgical energy.

In accordance with another aspect, the present invention provides a method for monitoring distances for formation of lesions at a plurality of axially spaced levels of tissue, the method comprising:
measuring a distance from a fixed reference to a designated region inside the patient; and
inputting the measured distance to a controller, wherein a graphic display is configured to provide an indication of a distance of electrodes of an electrosurgical device from the fixed reference based on an inputting of the measured distance.

In some embodiments, inputting the measured distance to the controller includes the step of adjusting a preset value stored by the controller to match the measured distance. In some embodiments, the adjustment of the preset value is disabled during application of electrosurgical energy.

Further features and advantages of the inventions are set forth in the following Description and Drawings, as well as in the appended claims.

### BRIEF DESCRIPTION OF THE DRAWINGS

FIG. 1 is a diagrammatic view of a unified system usable in association with a family of different treatment devices for treating body sphincters and adjoining tissue regions in different regions of the body.

FIG. 2 is a perspective view, with portions broken away, of one type of treatment device usable in association with the system shown in FIG. 1 to treat tissue in the upper gastro-intestinal tract, the treatment device having an operative element for contacting tissue shown in a collapsed condition.

FIG. 3 is a perspective view, with portions broken away, of the device shown in FIG. 2, with the operative element shown in an expanded condition.

FIG. 4 is a perspective view, with portions broken away, of the device shown in FIG. 2, with the operative element shown in an expanded condition and the electrodes extended for use.

FIG. 5 is a lesion pattern that can be formed by manipulating the device shown FIGS. 2 to 4 in the esophagus at or near the lower esophageal sphincter and in the cardia of the stomach, comprising a plurality of axially spaced lesion levels, each lesion level comprising a plurality of circumferential spaced lesions.

FIG. 6 is a perspective view of another type of treatment device usable in association with the system shown in FIG. 1 to treat tissue in the lower gastrointestinal tract, the treatment device having an array of electrodes shown in a retracted position.

FIG. 7 is a perspective view of the device shown in FIG. 6, with the array of electrodes shown in their extended position.

FIG. 8 is a perspective view of the device shown in FIGS. 6 and 7, with the array of electrodes shown in their extended position deployed in the lower gastrointestinal tract to treat sphincter dysfunction in the anal canal.

FIG. 9 is a lesion pattern that can be formed by manipulating the device as shown FIG. 8 in the anal canal at or near the anal sphincter, comprising a plurality of axially spaced lesion levels, each lesion level comprising a plurality of circumferential spaced lesions.

FIGS. 10A and 10B are, respectively, left and right perspective views of one embodiment of an integrated device incorporating features of the system shown in FIG. 1 and usable with either treatment device shown in FIG. 2 or 6 for treating body sphincters and adjoining tissue regions, and also having a controller and a graphical user display for visually prompting a user in a step-wise fashion to use a treatment device to perform a process of forming a pattern of lesions in a body region like that shown in FIG. 5 or 9 to guide the user so that individual lesions desired within a given level are all formed, and that a given level of lesions is not skipped.

FIG. 10C is a perspective view of an alternate embodiment of an integrated device incorporating features of the system shown in FIG. 1;

FIG. 11 is a representative graphical user set-up display generated by the controller prompting the user with numbers and/or text and/or icons through the set-up and connection steps prior to a treatment procedure.

FIG. 12 is a representative graphical user set-up display generated by the controller upon identifying the connection of a device like that shown in FIGS. 2 to 4 (identified by the trademark STRETTA®).

FIG. 13 is a representative graphical user set-up display generated by the controller upon identifying the connection of a device like that shown in FIGS. 6 to 8 (identified by the trademark SECCA®).

FIGS. 14-A to 14-O are representative graphical user treatment displays generated by the controller for visually prompting a user to use a treatment device like that shown in FIGS. 2 to 4 in a step-wise fashion to perform a process of forming a pattern of lesions in an esophagus like that shown in FIG. 5, the graphical user display guiding the user and creating a visual record of the progress of the process from start to finish, so that individual lesions desired within a given level are all formed, and that a given level of lesions is not skipped.

FIGS. 15A to 15I are representative graphical user treatment displays generated by the controller for visually prompting a user to use a treatment device like that shown in FIGS. 6 to 8 in a step-wise fashion to perform a process of forming a pattern of lesions in an anal canal like that shown in FIG. 9, the graphical user display guiding the user and creating a visual record of the progress of the process from start to finish, so that individual lesions desired within a given level are all formed, and that a given level of lesions is not skipped.

FIGS. 16A to 16D are representative graphical user treatment displays generated by the controller, in accordance with an alternate embodiment of the present invention, for visually prompting a user to use a treatment device like that shown in FIGS. 2 to 4 in a step-wise fashion to perform a process of forming a pattern of lesions in an esophagus like that shown in FIG. 5, the graphical user display guiding the user and creating a visual record of the progress of the process from start to finish, so that individual lesions desired within a given level are all formed, and that a given level of lesions is not skipped and further numerically indicating the distance from a fixed reference outside the patient.

FIGS. 17A to 17E are representative graphical user treatment displays generated by the controller, in accordance with an alternate embodiment of the present invention, for visually prompting a user to use a treatment device like that shown in FIGS. 6 to 8 in a step-wise fashion to perform a process of forming a pattern of lesions in an anal canal like that shown in FIG. 9, the graphical user display guiding the user and creating a visual record of the progress of the process from start to finish, so that individual lesions desired within a given level are all formed, and that a given level of lesions is not skipped and further numerically indicating the distance from a fixed reference.

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DESCRIPTION OF PREFERRED EMBODIMENTS

This Specification discloses various systems and methods for treating dysfunction of sphincters and adjoining tissue regions in the body. The systems and methods are particularly well suited for treating these dysfunctions in the upper and lower gastrointestinal tract, e.g., gastro-esophageal reflux disease (GERD) affecting the lower esophageal sphincter and adjacent cardia of the stomach, or fecal incontinence affecting the internal and external sphincters of the anal canal. For this reason, the systems and methods will be described in this context. Still, it should be appreciated that the disclosed systems and methods are applicable for use in treating other dysfunctions elsewhere in the body, and dysfunctions that are not necessarily sphincter-related. For example, the various aspects of the invention have application in procedures requiring treatment of hemorrhoids, or urinary incontinence, or restoring compliance to or otherwise tightening interior tissue or muscle regions. The systems and methods that embody features of the invention are also adaptable for use with systems and surgical techniques that are catheter-based and not necessarily catheter-based.

### I. Overview of the System

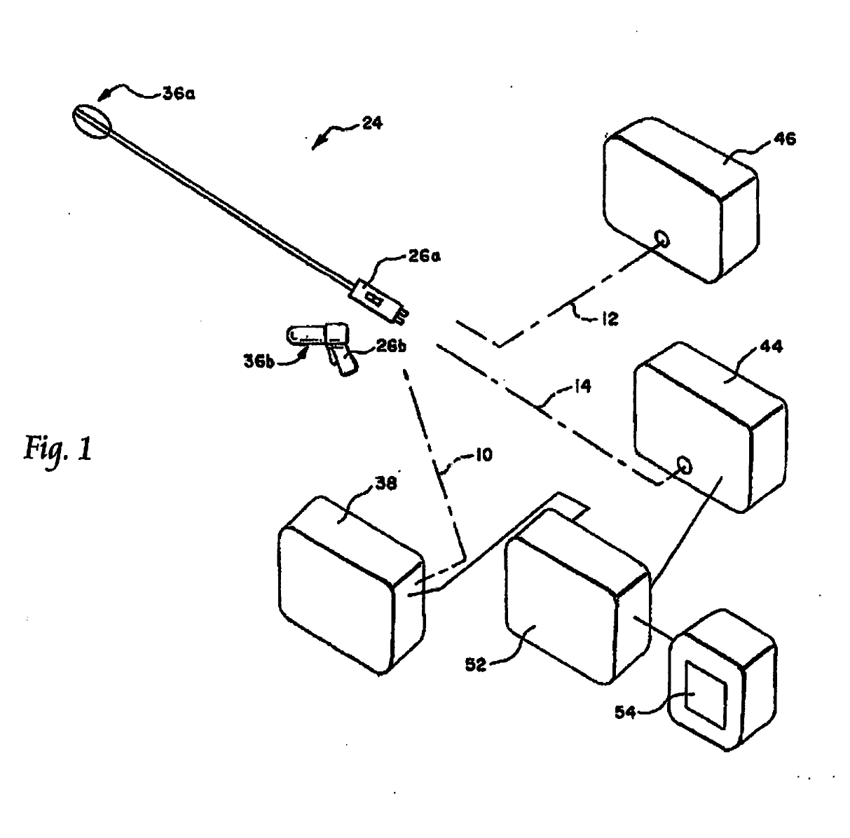

FIG. 1 shows a unified system 24 for diagnosing and/or treating dysfunction of sphincters and adjoining tissue in different regions of the body. In the illustrated embodiment, the system 24 is configured to diagnose and treat dysfunction in at least two distinct sphincter regions within the body.

The targeted sphincter regions can vary. In the illustrated embodiment, one region comprises the upper gastro-intestinal tract, e.g., the lower esophageal sphincter and adjacent cardia of the stomach. The second region comprises the lower gastrointestinal tract, e.g., in the intestines, rectum and anal canal.

The system 24 includes a family of treatment devices 26a and 26b. Each device 26a and 26b can be specifically configured according to the physiology and anatomy of the particular sphincter region which it is intended to treat. The details of construction of each device 26a and 26b will be generally described later for purposes of illustration.

Each device 26a/26b carries an operative element 36a and 36b. The operative element 36a and 36b can be differently configured according to the physiology and anatomy of the particular sphincter region which it is intended to treated. Still, if the anatomy and physiology of the two treatment regions are the same or similar enough, the configuration of the operative elements 36a and 36b can be same or essentially the same.

In the illustrated embodiment, the operative elements 36a and 36b function in the system 10 to apply energy in a selective fashion to tissue in or adjoining the targeted sphincter region. The applied energy creates one or more lesions, or a prescribed pattern of lesions, below the surface of the targeted region. The subsurface lesions are desirably formed in a manner that preserves and protects the surface against thermal damage.

Natural healing of the subsurface lesions leads to a physical tightening of the targeted tissue. The subsurface lesions can also result in the interruption of aberrant electrical pathways that may cause spontaneous sphincter relaxation. In any event, the treatment can restore normal closure function to the sphincter region 18.

The system 24 includes a generator 38 to supply the treatment energy to the operative element 36a/36b of the device 26a/26b selected for use. In the illustrated embodiment, the generator 38 supplies radio frequency energy, e.g., having a frequency in the range of about 400 kHz to about 10 mHz. Of course, other forms of energy can be applied, e.g., coherent or incoherent light; heated or cooled fluid; resistive heating; microwave; ultrasound; a tissue ablation fluid; or cryogenic fluid.

A selected device 26a/26b can be individually coupled to the generator 38 via a cable 10 to convey the generated energy to the respective operative element 36a/36b.

The system 24 preferably also includes certain auxiliary processing equipment. In the illustrated embodiment, the processing equipment comprises an external fluid delivery apparatus 44 and an external aspirating apparatus 46.

A selected device 26a/26b can be connected via tubing 12 to the fluid delivery apparatus 44, to convey processing fluid for discharge by or near the operative element 36a/36b. A selected device 26a/26b can also be connected via tubing 14 to the aspirating apparatus 46, to convey aspirated material from or near from the operative element 36a/36b for discharge.

The system 24 also includes a controller 52. The controller 52, which preferably includes a central processing unit (CPU), is linked to the generator 38, the fluid delivery apparatus 44, and the aspirating apparatus 46. Alternatively, the aspirating apparatus 46 can comprise a conventional vacuum source typically present in a physician's suite, which operates continuously, independent of the controller 52.

The controller 52 governs the power levels, cycles, and duration that the radio frequency energy is distributed to the particular operative element 36a/36b, to achieve and maintain power levels appropriate to achieve the desired treatment objectives. In tandem, the controller 52 also desirably governs the delivery of processing fluid and, if desired, the removal of aspirated material.

The controller 52 includes an input/output (I/O) device 54. The I/O device 54 allows the physician to input control and processing variables, to enable the controller to generate appropriate command signals. The I/O device 54 also receives real time processing feedback information from one or more sensors associated with the operative element (as will be described later), for processing by the controller 52, e.g., to govern the application of energy and the delivery of processing fluid.

The I/O device 54 also includes a graphical user interface (GUI), to graphically present processing information to the physician for viewing or analysis. Further details regarding the GUI will be provided later.

### II. The Treatment Devices

The structure of the operative element 36 can vary. Various representative embodiments will be described.

A. For Treatment of Upper Gastro-Intestinal Tract

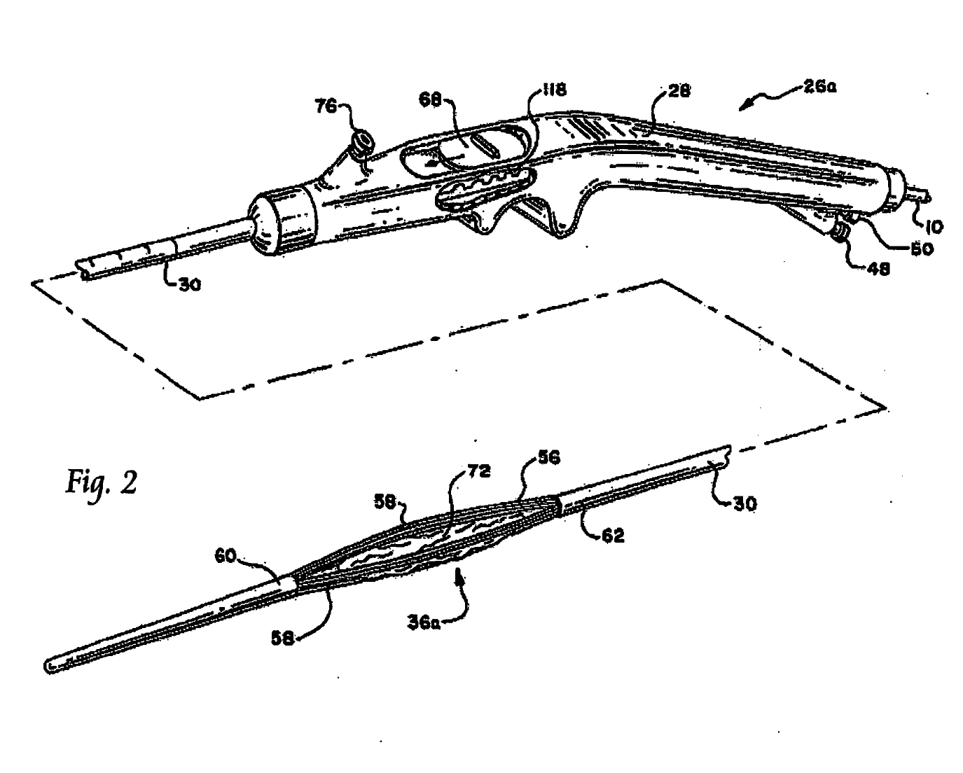

FIGS. 2 to 4 show a catheter-based device 26a for treating sphincter regions in the upper gastro-intestinal tract, and more particularly, the lower esophageal sphincter and adjoining cardia of the stomach to treat GERD. In the embodiment shown, the device 26a includes a flexible catheter tube 30 that carries a handle 28 at its proximal end. The distal end of the catheter tube 30 carries the operative element 36a.

In the illustrated embodiment, the operative element 36a comprises a three-dimensional basket 56. The basket 56 includes one or more spines 58, and typically includes from four to eight spines 58, which are assembled together by a distal hub 60 and a proximal base 62. In the illustrated embodiment, four spines 58 are shown, spaced circumferentially at 90-degree intervals In the illustrated embodiment, an expandable structure 72 comprising a balloon is located within the basket 56. The balloon structure 72 can be made, e.g., from a Polyethylene Terephthalate (PET) material, or a polyamide (non-compliant) material, or a radiation cross-linked polyethylene (semi-compliant) material, or a latex material, or a silicone material, or a C-Flex (highly compliant) material.

The balloon structure 72 presents a normally, generally collapsed condition, as FIG. 2 shows. In this condition, the basket 56 is also normally collapsed about the balloon structure 72, presenting a low profile for deployment into the esophagus.

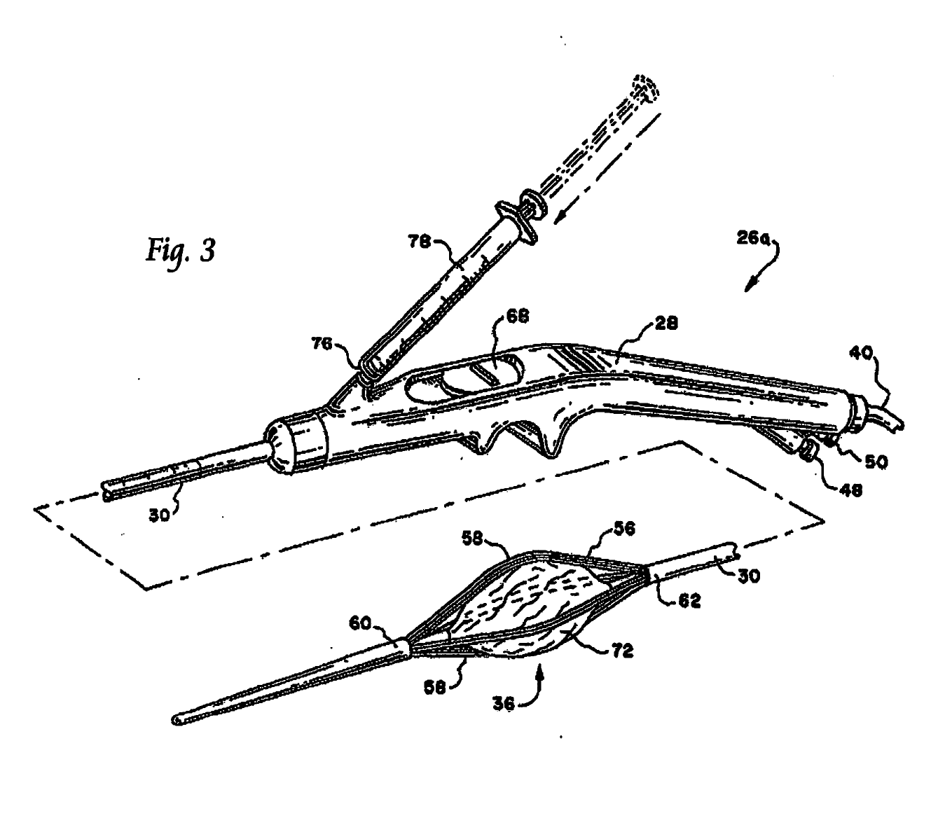

A catheter tube 30 includes an interior lumen, which communicates with the interior of the balloon structure 72. A fitting 76 (e.g., a syringe-activated check valve) is carried by the handle 28. The fitting 76 communicates with the lumen. The fitting 76 couples the lumen to a syringe 78 (see FIG. 3). The syringe 78 injects fluid under pressure through the lumen into the balloon structure 72, causing its expansion.

Expansion of the balloon structure 72 urges the basket 56 to open and expand (see FIG. 3). The force exerted by the balloon structure 72, when expanded, is sufficient to exert an opening or dilating force upon the tissue surrounding the basket 56 (see FIG. 31).

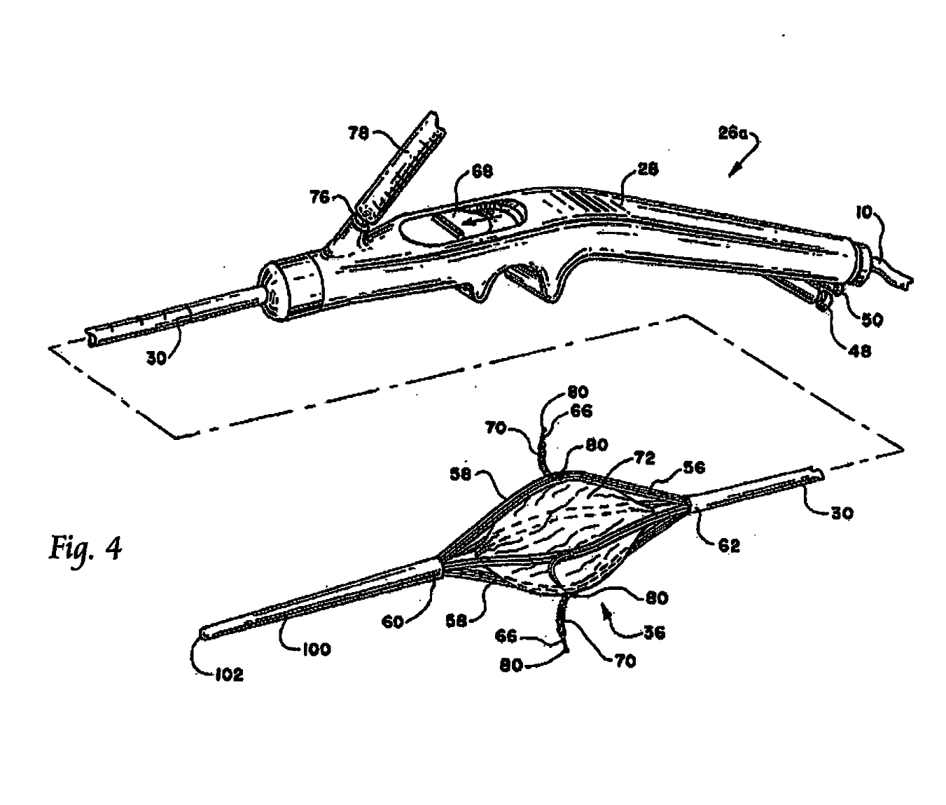

Each spine 58 carries an electrode 66 (see FIG. 4). Therefore, there are four electrodes circumferentially spaced at 90-degree intervals. In the illustrated embodiment, each electrode 66 is carried within the tubular spine 58 for sliding movement. Each electrode 66 slides from a retracted position, withdrawn in the spine 58 (shown in FIG. 3) and an extended position, extending outward from the spine 58 (see FIG. 4) through a hole in the spine 58. A push-pull lever 68 on the handle 28 is coupled by one or more interior wires to the sliding electrodes 66. The lever 68 controls movement of the electrodes between the retracted position (by pulling rearward on the lever 68) and the extended position (by pushing forward on the lever 68).

The electrodes 66 have sufficient distal sharpness and strength, when extended, to penetrate a desired depth into tissue the smooth muscle of the lower esophageal sphincter 18 or the cardia of the stomach 16 (see FIG. 32). The desired depth can range from about 4 mm to about 5 mm.

The electrodes 66 are formed of material that conducts radio frequency energy, e.g., nickel titanium, stainless steel, e.g., 304 stainless steel, or a combination of nickel titanium and stainless steel.

In the illustrated embodiment (see FIG. 4), an electrical insulating material 70 is coated about the proximal end of each electrode 66. When the distal end of the electrode 66 penetrating the smooth muscle of the esophageal sphincter 18 or cardia 20 transmits radio frequency energy, the material 70 insulates the mucosal surface of the esophagus 10 or cardia 20 from direct exposure to the radio frequency energy. Thermal damage to the mucosal surface is thereby avoided. The mucosal surface can also be actively cooled during application of radio frequency energy, to further protect the mucosal surface from thermal damage.

In the illustrated embodiment (see FIG. 4), at least one temperature sensor 80 is associated with each electrode. One temperature sensor 80 senses temperature conditions near the exposed distal end of the electrode 66, a second temperature sensor 80 is located on the corresponding spine 58, which rests against the mucosal surface when the balloon structure 72 is inflated.

The external fluid delivery apparatus 44 is coupled via tubing 12 (see FIG. 1) to connector 48 (see FIG. 4), to supply cooling liquid to the targeted tissue, e.g., through holes in the spines. The external aspirating apparatus 46 is coupled via tubing 14 (see FIG. 1) to connector 50 (see FIG. 4), to convey liquid from the targeted tissue site, e.g., through other holes in the spine or elsewhere on the basket 56. The controller 52 can govern the delivery of processing fluid and, if desired, the removal of aspirated material.

The controller 52 can condition the electrodes 66 to operate in a monopolar mode. In this mode, each electrode 66 serves as a transmitter of energy, and an indifferent patch electrode (described later) serves as a common return for all electrodes 66. Alternatively, the controller 52 can condition the electrodes 66 to operate in a bipolar mode. In this mode, one of the electrodes comprises the transmitter and another electrode comprises the return for the transmitted energy. The bipolar electrode pairs can be electrodes 66 on adjacent spines, or electrodes 66 spaced more widely apart on different non-adjacent spines.

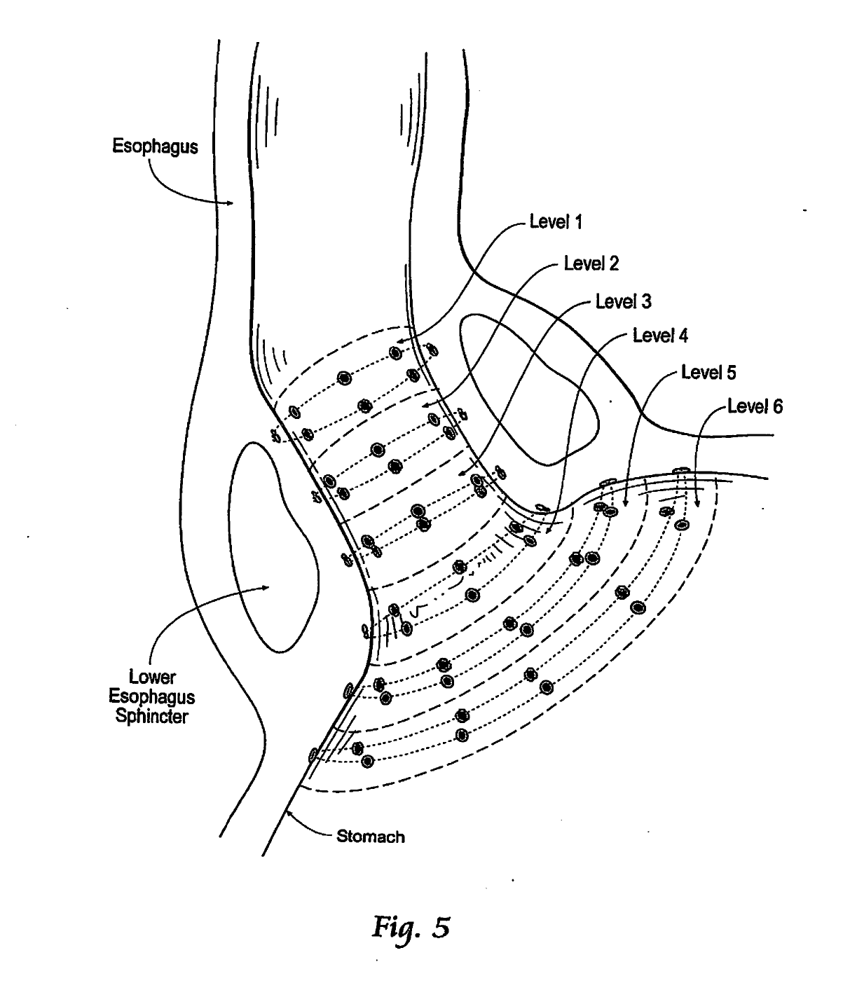

In use, the device 26a is manipulated to create a preferred pattern of multiple lesions comprising circumferential rings of lesions at several axially spaced-apart levels (about 5 mm apart), each level comprising from 8 to 12 lesions. A representative embodiment of the lesion pattern is shown in FIG. 5. As FIG. 5 shows, the rings are preferably formed in the esophagus in regions above the stomach, at or near the lower esophageal sphincter, and/or in the cardia of the stomach. The rings in the cardia are concentrically spaced about the opening funnel of the cardia. At or near the lower esophageal sphincter, the rings are axially spaced along the esophagus.

Multiple lesion patterns can be created by successive extension and retraction of the electrodes 66, accompanied by rotation and/or axial movement of the catheter tube to reposition the basket 56. The physician can create a given ring pattern by expanding the balloon structure 72, extending the electrodes 66 at the targeted treatment site and applying energy, to form a first set of four lesions. The physician can then withdraw (retract) the electrodes 66, collapse the balloon structure 72, and rotate the catheter tube 30 by a desired amount, e.g., 30-degrees or 45-degrees, depending upon the number of total lesions desired within 360-degrees. The physician can then again expand the structure 72 and again extend the electrodes 66 and apply energy, to achieve a second set of four lesions. The physician repeats this sequence until a desired number of lesions within the 360-degree extent of the ring is formed. Additional lesions can be created at different levels by advancing the operative element axially, gauging the ring separation by external markings on the catheter tube.

As shown in FIG. 5, a desirable pattern comprises an axially spaced pattern of six circumferential lesions numbered Level 1 to Level 6 in an inferior direction, with some layers in the cardia of the stomach, and others in the esophagus above the stomach at or near the lower esophageal sphincter. In the embodiment shown in instant FIG. 5, in the Levels 1, 2, 3, and 4, there are eight lesions circumferentially spaced 45-degrees apart (i.e., a first application of energy, followed by a 45-degree rotation of the basket 56, followed by a second application of energy). In the Levels 5 and 6, there are twelve lesions circumferentially spaced 30-degrees apart (i.e., a first application of energy, followed by a 30-degree rotation of the basket 56, followed by a second application of energy, followed by a 30-degree rotation of the basket 56, followed by a third application of energy). In Level 5, the balloon structure 72 is only partially expanded, whereas in Level 6, the balloon structure 72 is more fully expanded, to provide lesion patterns that increase in circumference according to the funnel-shaped space available in the funnel of the cardia.

B. For Treatment of Lower Gastro-Intestinal Tract

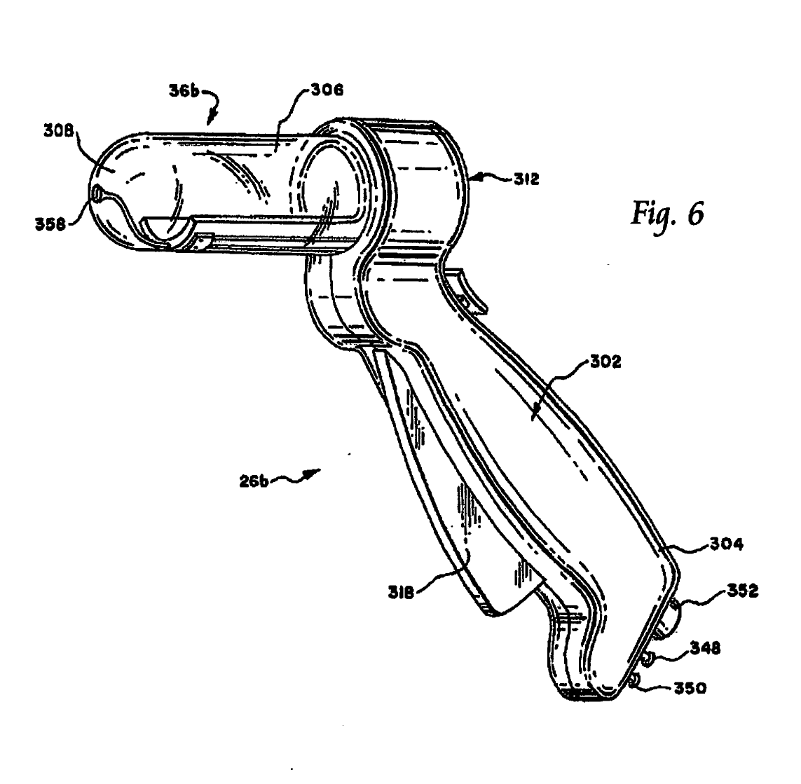

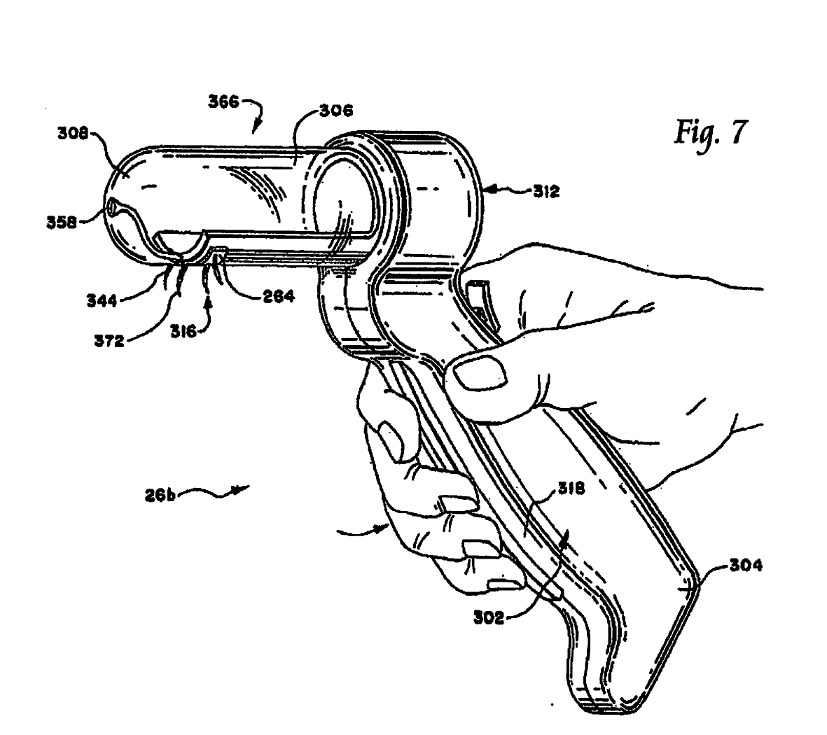

FIGS. 6 and 7 show a representative embodiment for device 26b, which takes the form of a hand manipulated device 302 for treating sphincter regions in the lower gastro-intestinal tract, and more particularly, the internal and/or external sphincter muscles in the anal canal to treat fecal incontinence. The device 302 includes a hand grip 304 that carries the operative element 36b.

In the illustrated embodiment, the operative element 36b takes the form of a hollow, tubular barrel 306 made from a transparent, molded plastic material. The barrel 306 terminates with a blunt, rounded distal end 308 to aid passage of the barrel 306 through the anal canal, without need for a separate introducer. The hand grip 304 includes a viewing port 312 for looking into the transparent, hollow interior of the barrel 306, to visualize surrounding tissue.

An array of needle electrodes 316 are movably contained in a side-by-side relationship along an arcuate segment of the barrel 306. In the illustrated embodiment, the needle electrodes 316 occupy an arc of about 67.5 degrees on the barrel 306. The needle electrodes 316 are mechanically linked to a finger-operated pull lever 318 on the hand grip 304. By operation of the pull lever 318, the distal ends of the needle electrodes 316 are moved between a retracted position (FIG. 5) and an extended position (FIG. 6 of the '523 patent). An electrical insulating material 344 is coated about the needle electrodes 316 (see FIG. 6 of the '523 patent), except for a prescribed region of the distal ends, where radio frequency energy is applied to tissue. The generator 38 is coupled via the cable 10 to a connector 352, to convey radio frequency energy to the electrodes 316.

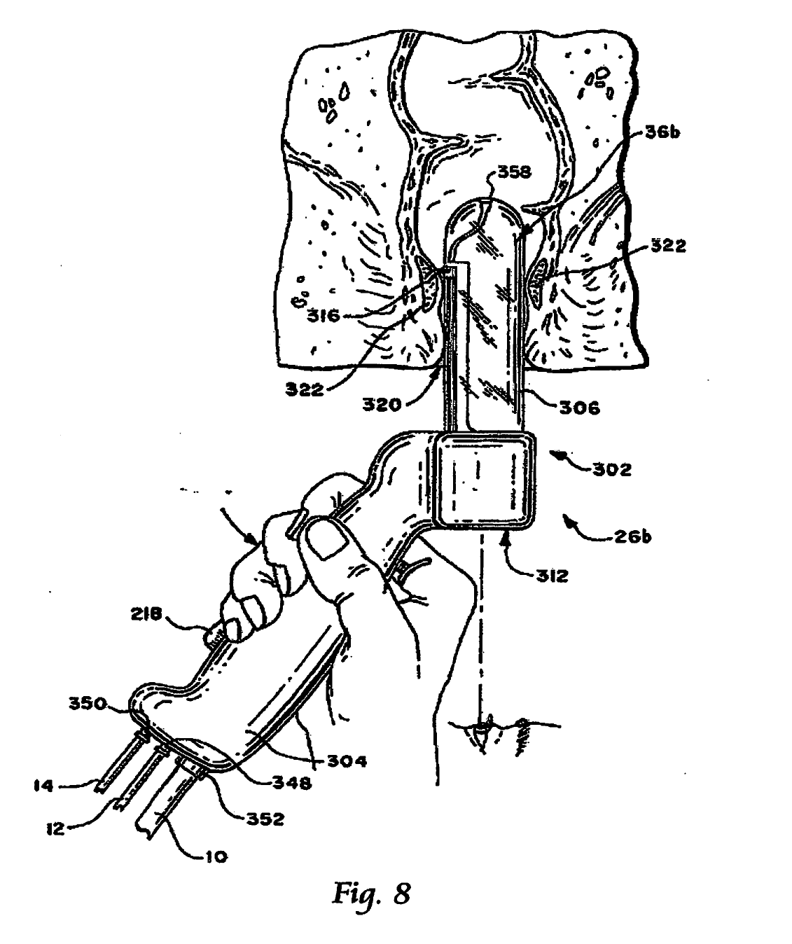

In use (see FIG. 8), the physician grasps the hand grip 304 and guides the barrel 306 into the anal canal 320. The pull lever 318 is in the neutral position and not depressed, so the needle electrodes 316 occupy their normal retracted position. Looking through the viewing port 312, the physician visualizes the pectinate (dentate) line through the barrel 306. Looking through the barrel 306, the physician positions the distal ends of the needle electrodes 316 at a desired location relative to the pectinate (dentate) line. A fiberoptic can also be located in the barrel 306 to provide local illumination. Once the distal end of the barrel 306 is located at the targeted site, the physician depresses the pull lever 318 (as FIG. 8 shows). The needle electrodes 316 advance to their extended positions. The distal ends of the electrodes 316 pierce and pass through the mucosal tissue into the muscle tissue of the target sphincter muscle. In FIG. 8, the distal end of the electrodes 316 are shown penetrating the involuntary, internal sphincter muscle 322. The physician commands the controller 52 to apply radio frequency energy through the needle electrodes 316. The energy can be applied simultaneously by all electrodes 316, or in any desired sequence.

The external fluid delivery apparatus 44 is coupled via tubing 12 to a connector 348 to convey a cooling liquid, e.g., through holes in the barrel 306, to contact tissue at a localized position surrounding the electrodes 316. The external aspirating apparatus 46 is coupled via tubing 14 to a connector 350 to convey liquid from the targeted tissue site, e.g., through an aspiration port 358 in the distal end 308 of the barrel 306 (see FIGS. 6 and 7).

The barrel 306 (see FIG. 7) also preferably carries temperature sensor 364, one of which is associated with each needle electrode 316. The sensors 364 sense tissue temperature conditions in the region adjacent to each needle electrode 316. Preferably, the distal end of each needle electrode 316 also carries a temperature sensor 372 (see FIG. 7).

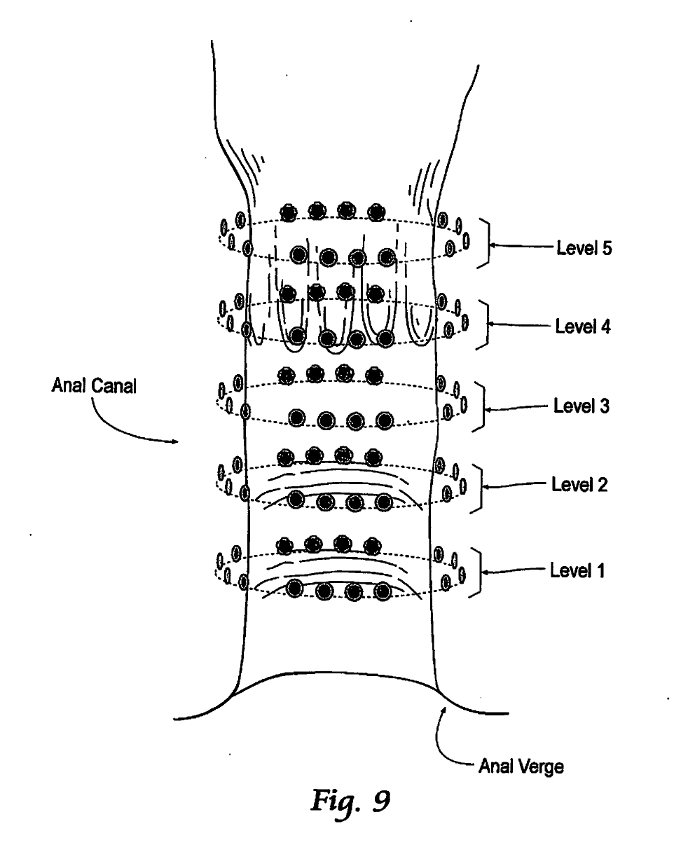

In use (see FIG. 9), a preferred pattern of multiple lesions is formed comprises several circumferential rings of lesions in axially spaced-apart levels (about 5 mm apart), each ring comprising 16 lesions in four quadrants of 4 each. The rings are formed axially along the anal canal, at or near the dentate line.

The fluid delivery apparatus 68 conveys cooling fluid for discharge at the treatment site, to cool the mucosal surface while energy is being applied by the needle electrodes 316. The aspirating apparatus 76 draws aspirated material and the processing fluid through the tubing 78 for discharge.

Referring to FIG. 9, the array of needle electrodes 316 is positioned at Level 1 to create four multiple lesions in the first quadrant. Upon the satisfactory creation of the lesion pattern in the first quadrant of Level 1, as just described, the physician actuates the button 64 to release the locking pawl 58 from the detent 62. The pull lever 52 returns to the spring-biased neutral position, thereby moving the needle electrodes 316 back to their retracted positions. Still grasping the hand grip 40 and visualizing through the viewing port 46, the physician moves the barrel 5 mm axially upward to Level 2, the first quadrant. The physician again deploys the needle electrodes 48 and performs another lesion generating sequence. The physician repeats this sequence of steps until additional number of lesion patterns are formed within the axially spaced first quadrants in Levels 1, 2, 3, 4, and 5.

Still grasping the hand grip 40 and visualizing through the viewing port 46, the physician returns to level 1, and rotates the barrel 42 a selected arcuate distance at the level of the first lesion pattern 94 to the second quadrant, i.e., by rotating the barrel 42 by ninety degrees.

The physician again deploys the needle electrodes 48 and performs another lesion generating sequence at quadrant 2 of Level 1. The physician then moves the barrel axially upward in 5 mm increments, at a number of axially spaced levels 2, 3, 4, and 5 generally aligned with lesion patterns 96, 98, and 100. Lesions are formed in this way in the second quadrant of Levels 1, 2, 3, 4, and 5.

The physician repeats the above described sequence two additional times, returning the barrel to level 1 and rotating the barrel 42 at successive intervals and axially repositioning the barrel 42 to form the lesion patterns quadrants 3 and 4 in the Levels 1, 2, 3, 4, and 5. This protocol forms a composite lesion pattern 102, which provides a density of lesions in the targeted sphincter tissue region to provoke a desired contraction of the sphincter tissue.

### III. System Operation

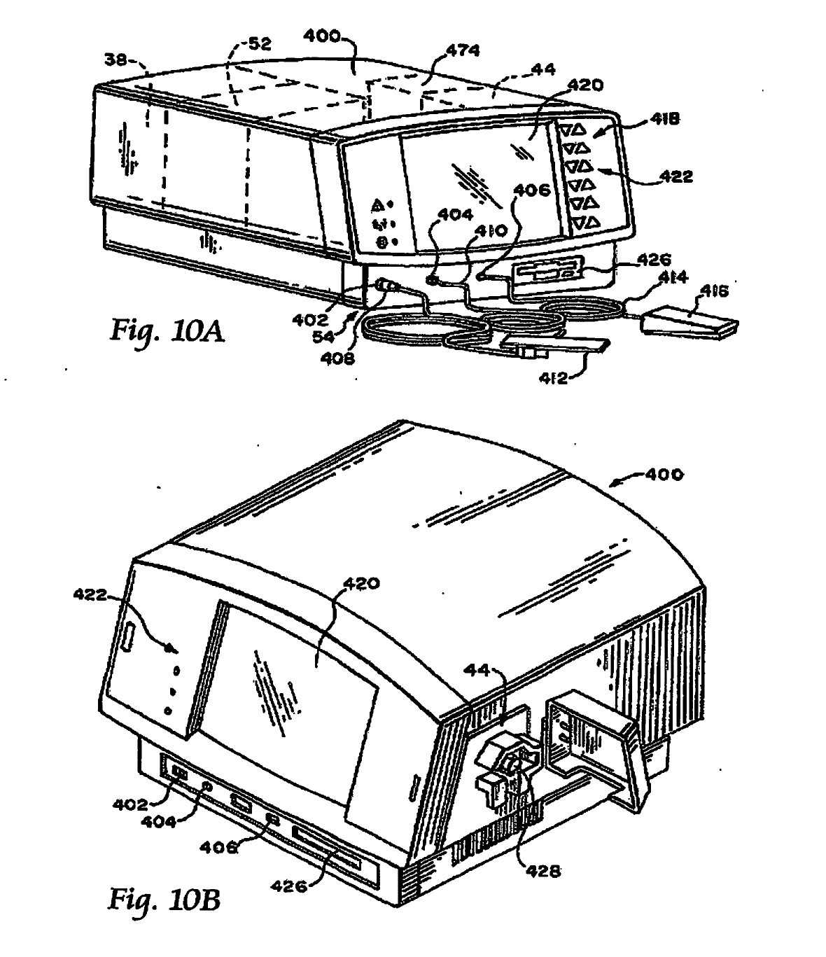

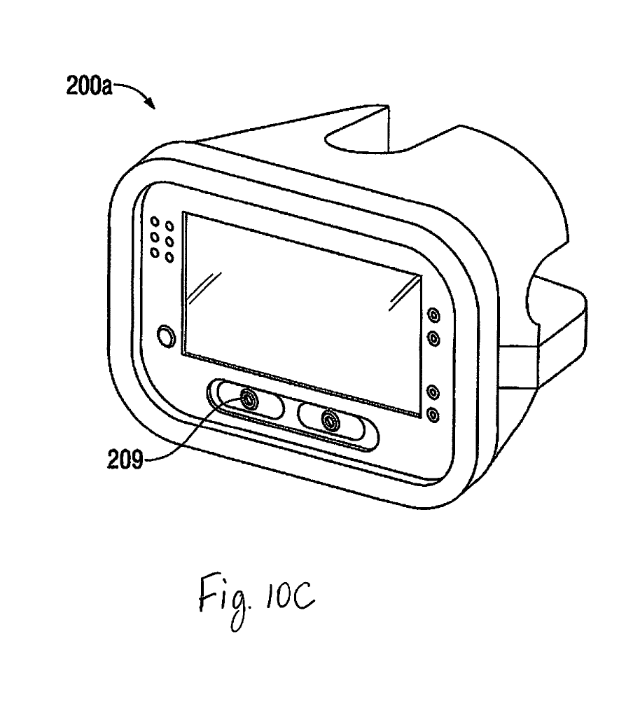

In the illustrated embodiment (see FIGS. 10A and 10B), the radio frequency generator 38, the controller 52 with I/O device 54, and the fluid delivery apparatus 44 (e.g., for the delivery of cooling liquid) are integrated within a single housing 400.

The I/O device 54 couples the controller 52 to a display microprocessor 474 (see FIG. 10A). The display microprocessor 474 is coupled to a graphics display monitor 420 in the housing 400. The controller 52 implements through the display microprocessor 474 the graphical user interface, or GUI, which is displayed on the display monitor 420. The graphical user interface is can be realized with conventional graphics software using the MS WINDOWS® application. The GUI 424 is implemented by showing on the monitor 420 basic screen displays.

FIG. 10C illustrates another embodiment where the radio frequency generator, the controller with I/O device, and the fluid delivery control apparatus (e.g., for the delivery of cooling liquid) are integrated within a single housing 200a. Connection port 209 is for connecting the treatment device.

A. Set-Up

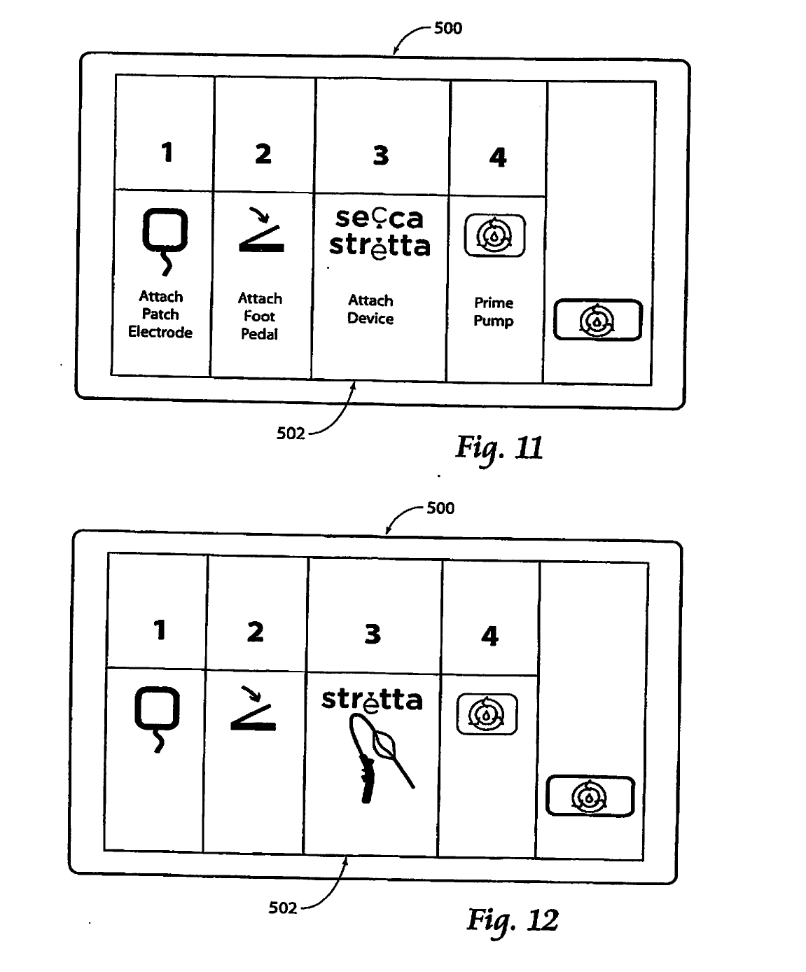

Upon boot-up of the CPU (see FIG. 11), the operating system implements the SET-UP function for the GUI 500. The GUI displays an appropriate start-up logo and title image (not shown), while the controller 52 performs a self-test. An array of SETUP prompts 502 leads the operator in a step-wise fashion through the tasks required to enable use of the generator and device. The physician can couple the source of cooling liquid to the appropriate port on the handle of the device 26a/26b (see FIG. 10A, as previously described) and load the tubing leading from the source of cooling liquid (e.g., a bag containing sterile water) into the pump rotor 428 (see FIG. 10B). The physician can also couple the aspiration source 46 to the appropriate port on the handle of the treatment device 26a/26b (as also already described). The physician can also couple the patch electrode 412 and foot pedal 416 (shown in FIG. 10A). In the SET-UP prompt array 502, a graphic field of the GUI 500 displays one or more icons and/or alpha-numeric indicia 502 that prompt the operator to connect the return patch electrode 412, connect the foot pedal or switch 416, connect the selected treatment device 26a (designed by its trademark STRETTA®) or 26b (designated by its trademark SECCA®), and to prime the irrigation pump 44.

The controller 52 ascertains which device 26a or 26b has been selected for use by reading a coded identification component residing in the handle of the device 26a or 26b. Based upon this input, the controller 52 proceeds to execute the preprogrammed control and graphical GUI command functions for the particular device 26a and 26b that is coupled to the generator.

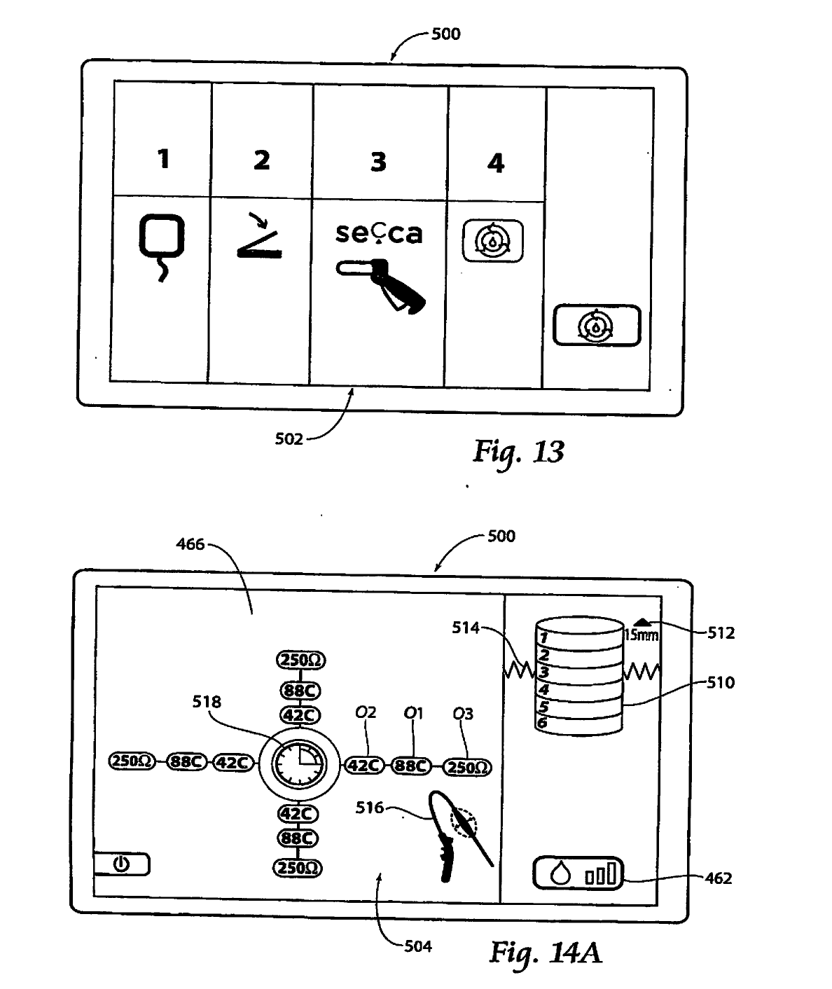

If the identification code for the device 26a, (STRETTA®) is registered, the GUI displays an appropriate start-up logo and title image for the device 26a (see FIG. 12). Likewise, if the identification code for the device 26b (SECCA®) is registered, the GUI displays an appropriate start-up logo and title image for the device 26b (FIG. 13).

B. Treatment Screens (UGUI and LGUI)

Upon completion of the SET-UP operation, the controller 52 proceeds to condition the generator and ancillary equipment to proceed step-wise through a sequence of operational modes. The operational modes have been preprogrammed to achieve the treatment protocol and objective of the selected device 26a/26b. The conduct of these operational modes and the appearance of the graphical user interface that guides and informs the user during the course of the selected procedure can differ between devices 26a and 26b.

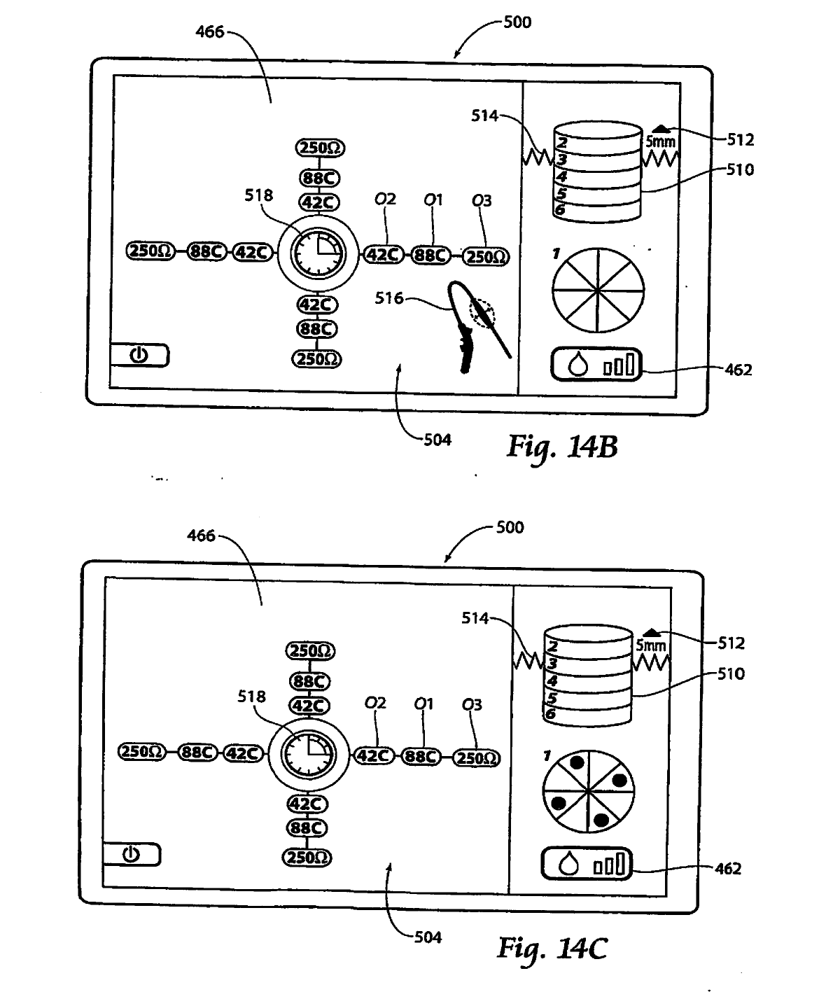

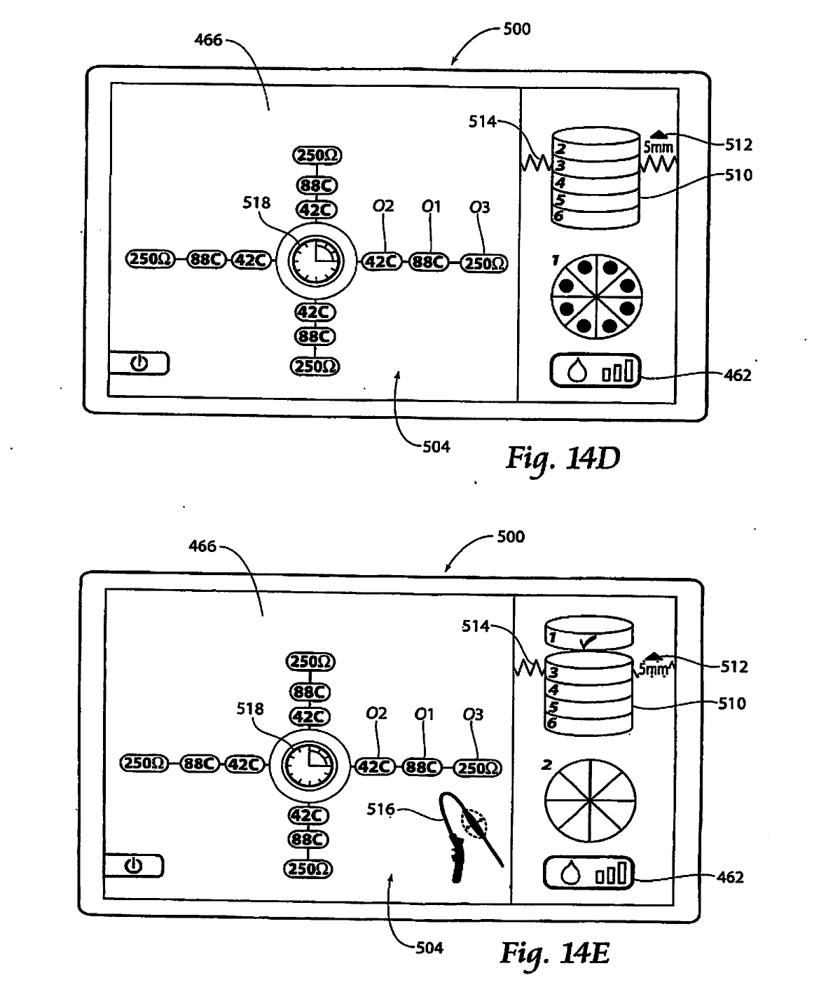

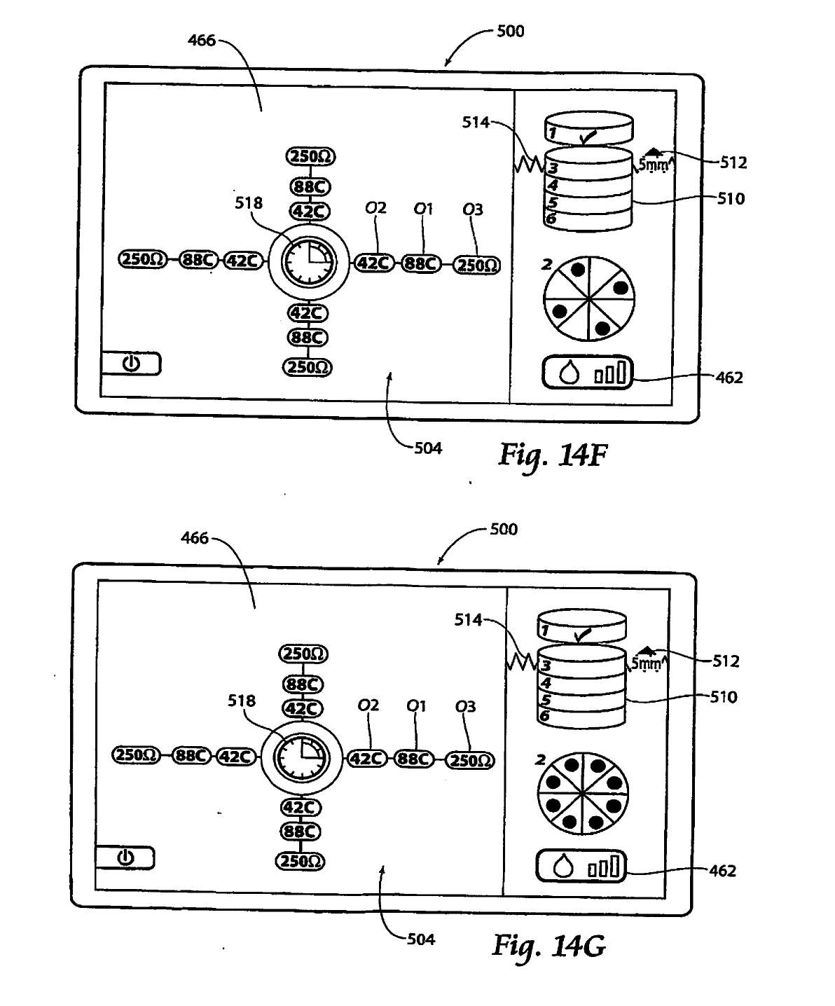

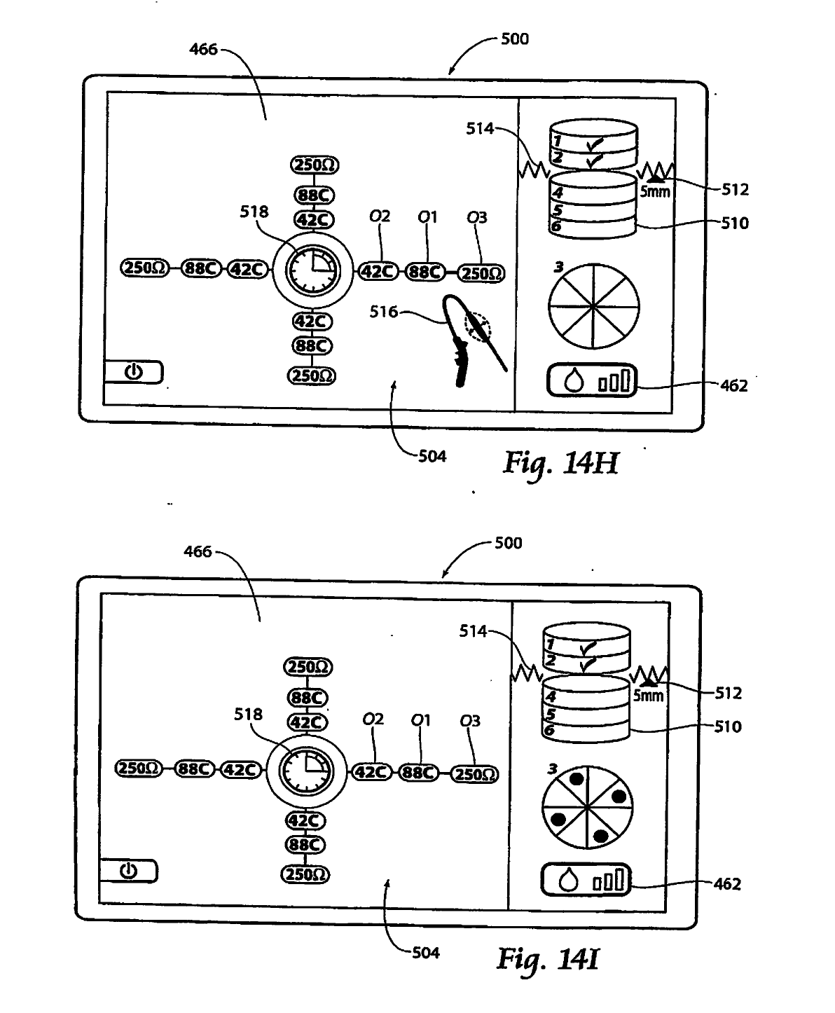

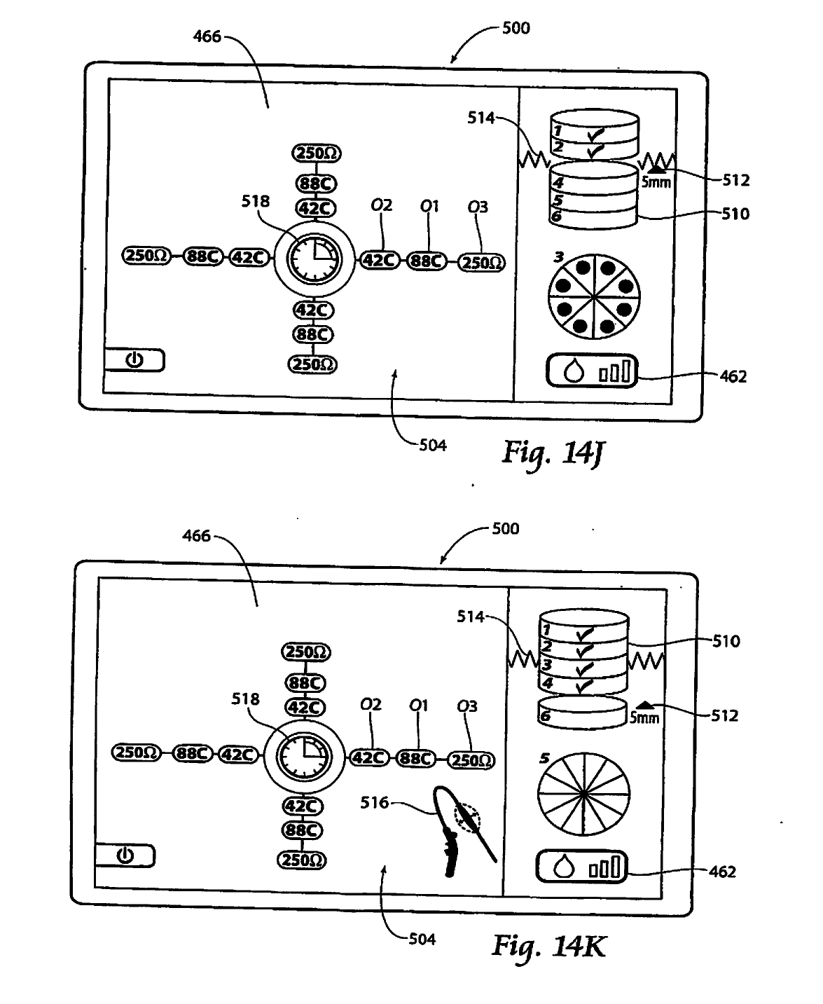

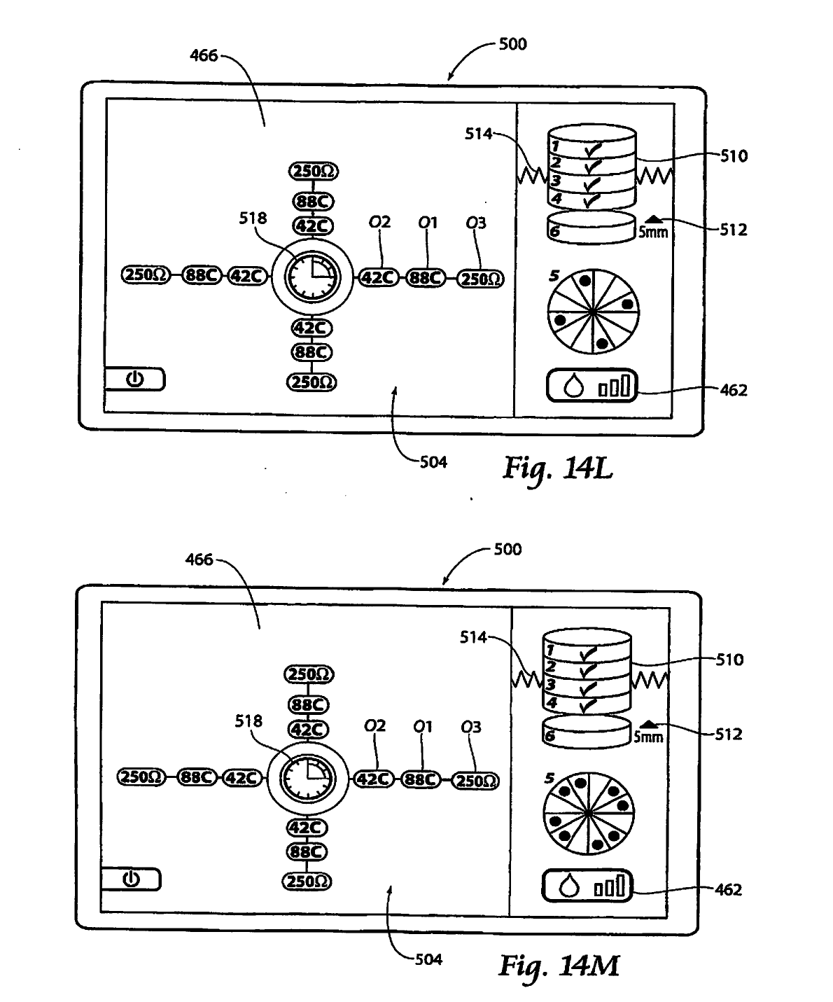

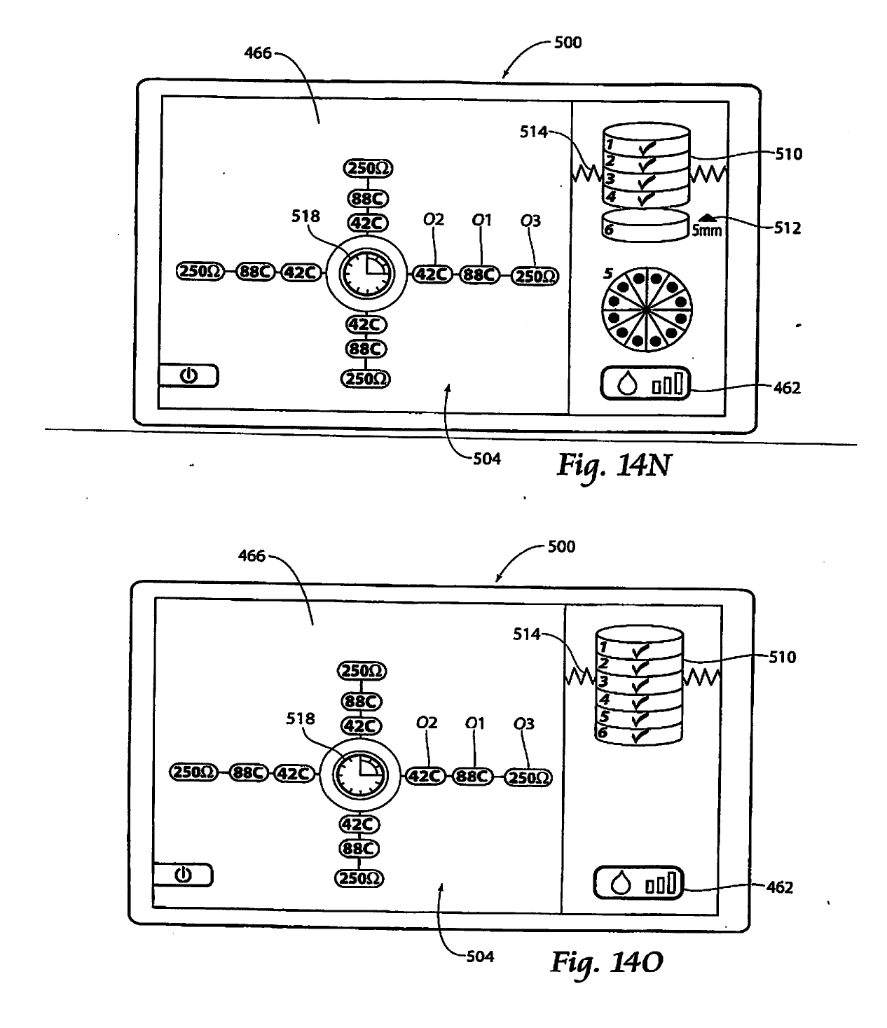

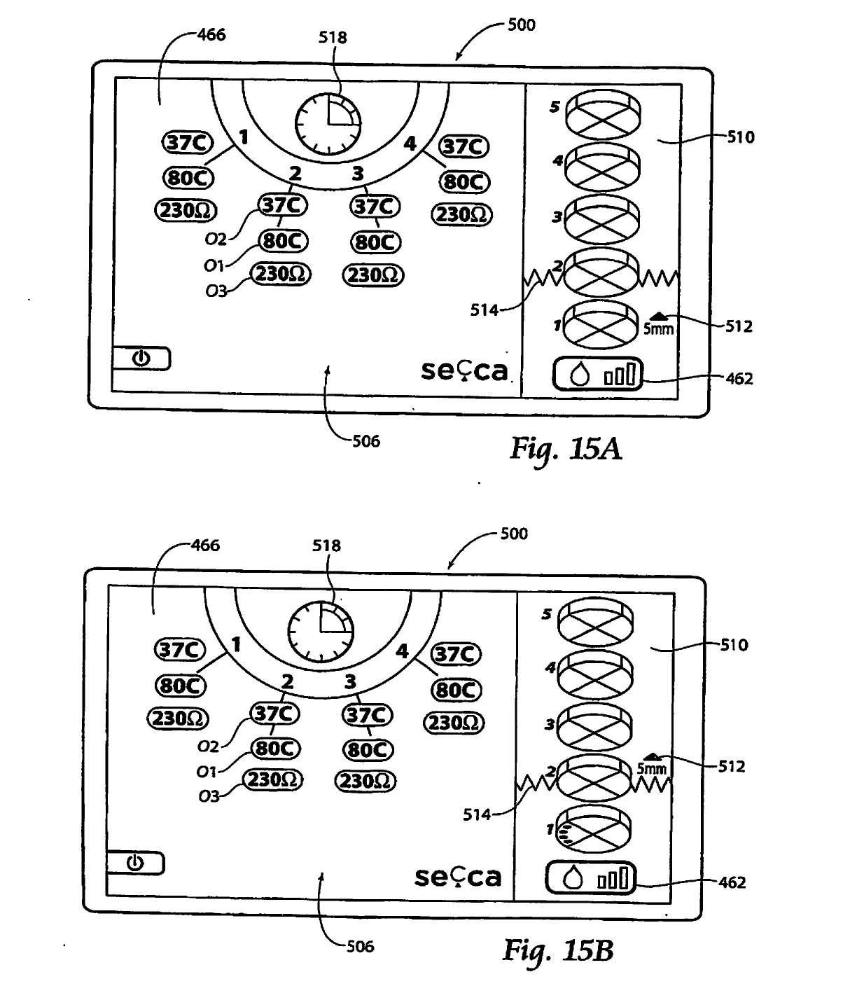

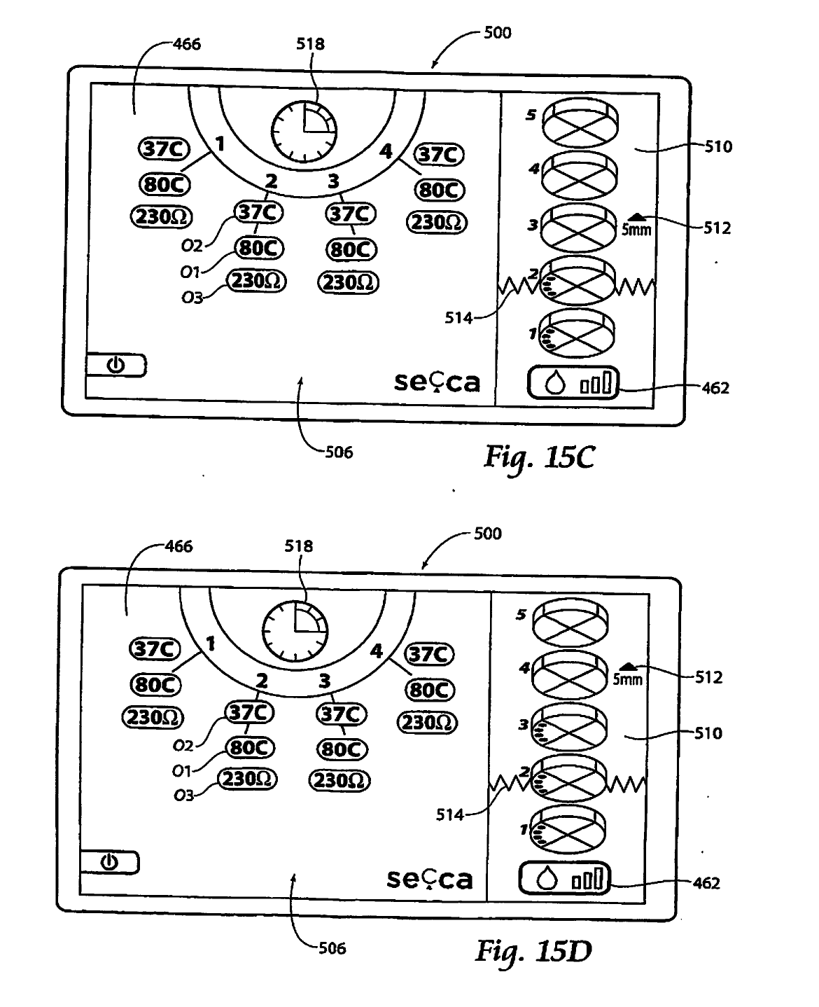

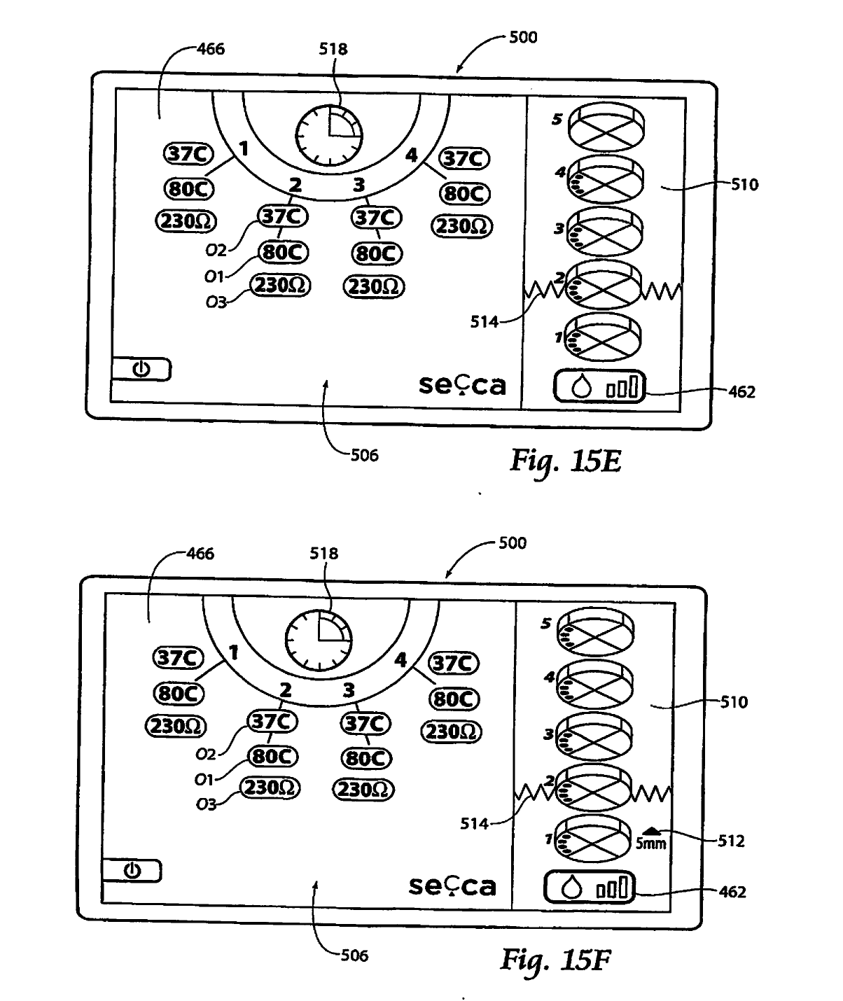

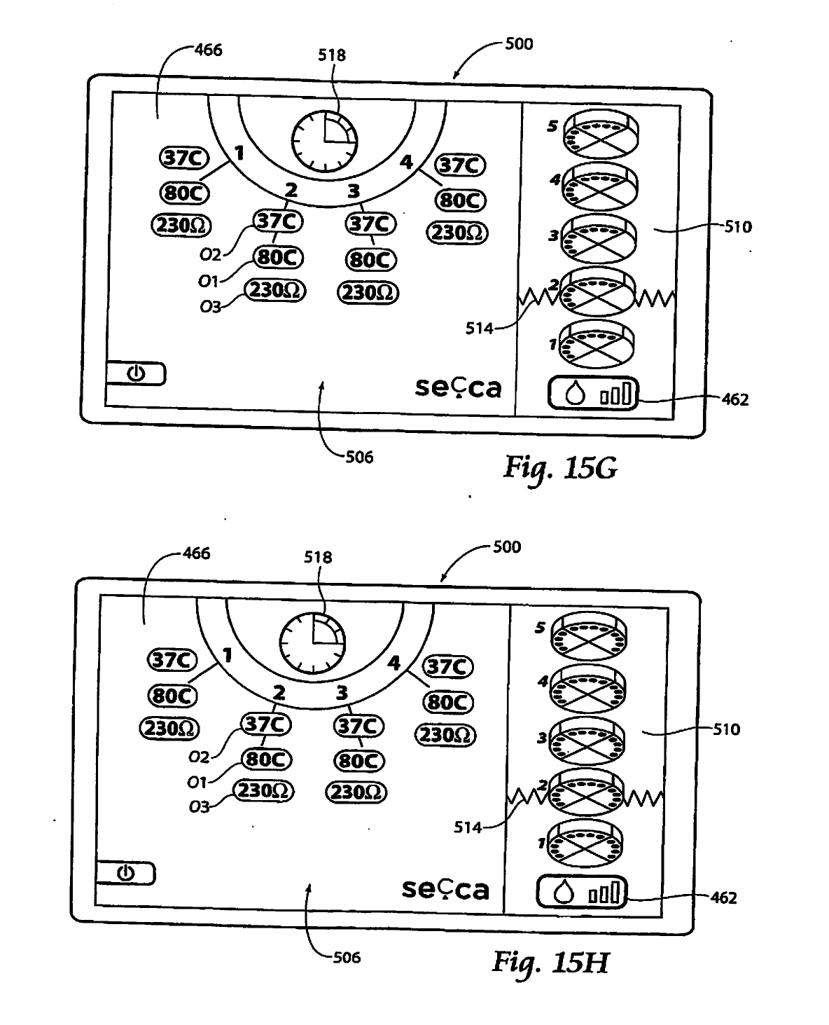

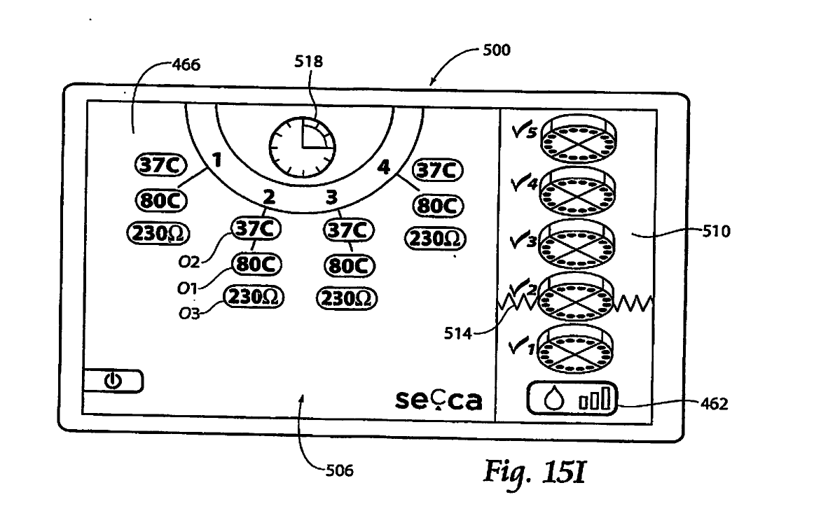

For ease of description, the GUI 500 displays for the upper gastro-intestinal procedure (i.e., for the device 26a) a treatment screen that will in shorthand be generally called UGUI 504 (FIG. 14A). Likewise, the GUI displays for the lower gastro-intestinal procedure (i.e., for the device 26b) a treatment screen that will in shorthand be generally called LGUI 506 (FIG. 15A).

In both the UGUI 504 (FIG. 14A) and LGUI 506 (FIG. 15A), there is a parameter icon 462 designating cooling fluid flow rate/priming. In both the UGUI 504 and the LGUI 506, the Flow Rate/Priming Icon 462 shows the selected pump speed by the number of bars, one bar highlighting a low speed, two bars highlighting a medium speed, and three bars highlighting a high speed.

Each UGUI 504 (FIG. 14A) and LGUI 506 (15A) includes an Electrode Icon 466. In general, each Electrode Icon 466 comprises an idealized graphical image, which spatially models the particular multiple electrode geometry of the treatment device 26a/26b that has been coupled to the controller 42. Just as the multiple electrode geometries of the devices 26a and 26b differ, so, too, does the Electrode Icon 466 of the UGUI 504 differ from the Electrode Icon 466 of the LGUI 506.

As FIG. 14A shows, in the UGUI 504, four electrodes are shown in the graphic image of the Icon 466, which are spaced apart by 90 degrees. This graphic image reflects the geometry of the four-electrode configuration of the device 26a, as shown in FIG. 4.

As FIG. 15A shows, in the LGUI 506, four electrodes are shown in the graphic image of Icon 466 in a circumferentially spaced relationship along a partial arcuate sector. This graphic image reflects the arrangement of electrodes on the treatment device 26b, as shown in FIG. 7.

For each electrode, the respective Icon 466 incorporates graphic regions O1, O2, and O3 in the spatial display. Regions O1 and O2 display temperature conditions encountered for that electrode. Region O1 numerically displays the mucosal layer temperature in UGUI 504 (FIG. 14A) and LGUI 506 (FIG. 15A). Region O2 numerically displays the electrode tip temperature for that electrode in UGUI 504 (FIG. 14A) and LGUI 506 (FIG. 15A). Region O3 displays the derived impedance value for each electrode. Both UGUI 504 and LGUI 506 display instantaneous, sensed temperature readings from the tip electrode and tissue surface, as well as impedance values, which are continuously displayed in spatial relation to the electrodes in the regions O1, O2, and O3.

The numeric displays of the regions O1/O2/O3 can be blanked out for a given electrode if the corresponding electrode/channel has been disabled, either by the physician or by a sensed out-of-bounds condition. An “acceptable” color indicator (e.g., green) can also be displayed in the background of the regions O1/O2/O3 as long as the sensed condition is within the desired pre-established ranges. However, if the sensed conditions fall outside the desired range, the color indicator changes to an “undesirable” color indicator (e.g., to grey), and numeric display is blanked out.

There is also a Lesion Level Icon 510 in each display UGUI 504 and LGUI 506, adjacent to the respective Electrode Icon 466. The Lesion Level Icon 510 comprises an idealized graphical image, which spatially models the desired lesion levels and the number of lesions in each level. Just as the lesion patterns created by the devices 26a and 26b differ, so, too, does the Lesion Level Icon 510 of the UGUI 504 differ from the Electrode Icon 466 of the LGUI 506.

As will be described in greater detail later, the Lesion Level Icons 510 change in real time, to step-wise guide the physician through the procedure and to record the progress of the procedure from start to finish. In many fundamental respects, the look and feel of the Lesion Level Icons 510 for the LGUI 504 and the LGUI 506 are similar, but they do differ in implantation details, due to the difference of the protocols of lesion formation.

Exemplary changes in the Lesion Level Icons 510 for the UGUI 504 and the LGUI 506 will now be described.

1. The UGUI

In the UGUI 504 (see FIG. 14A), six numbered Lesion Levels 1, 2, 3, 4, 5, and 6 are displayed, to correspond with the lesion levels already described and shown in FIG. 5. The UGUI 504 also displays a squiggle line 514, which marks where the physician has visualized a selected anatomic home base reference for the formation of lesions within the esophagus for treatment. Guided by the UGUI 504, lesions are placed relative to this anatomic home base.

In preparation for the treatment, the physician visualizes in the esophagus the Z-line or other desired anatomic landmark. Markers are arranged at 5 mm intervals along the catheter tube. Upon visualizing the Z-line, the physician notes the external marker on the catheter tube that corresponds to this position. With reference to the markers, the physician can then axially advance or retract the catheter tube in 5 mm increments, which correspond to the desired spacing between the lesion levels. This orientation of lesion levels is also shown in FIG. 5.

The UGUI 504 graphically orients the location of Lesion Levels 4, 5, and 6 relative to this anatomical base, displaying Lesion Levels either below (inferior to) the squiggle line 514 (Lesion Levels 4, 5, and 6) or at or above the squiggle line 514 (Lesion Levels 1, 2, and 3).

As will be described, the UGUI 504 graphically changes the display of the Lesion Levels, depending upon the status of lesion formation within the respective levels.

FIG. 14A shows a representative first graphical form of a given lesion level. The graphical form comprises, e.g., a cylinder that faces edgewise on the UGUI 504, as is shown for Lesion Levels 1 to 6 in FIG. 14A. This graphical form indicates at a glance that no lesions are present in the respective lesion levels.

As is shown in FIG. 14A, next to the graphical form of the edgewise cylinder of Lesion Level 1 is a Guide Marker 512. The Guide Marker 514 indicates that formation of lesions in Lesion Level 1 is the first to be indicated. A numeric value (15 mm) is displayed in association with the edgewise cylinder of Lesion Level 1, which indicates that Lesion Level 1 is 15 mm from the anatomic landmark. The orientation of Lesion Level 1 above (superior to) the squiggle line 514 guides the physical to advance the catheter tube upward from the anatomic marker by 15 mm, to place it at Lesion Level 1. A Balloon Icon 516 prompts the physician to expand the basket of the device 26a at Lesion Level 1.

Upon sensing electrode impedance, indicating contact with tissue at Lesion Level 1 (or in response to another input indicating deployment of the device 26a at the desired lesion level), the controller commands the UGUI 504 to change the graphical form of Lesion Level 1 to a second graphical form, which is shown in FIG. 14B. The second graphical form (shown in FIG. 14B) is different than the first graphical form (shown in FIG. 14A). The graphical form comprises, e.g., a segmented circle, with a numeric indicator next to it. This is shown for Lesion Level 1 in FIG. 14B. In visual effect, the second graphical form shows the previous cylinder form rotated for viewing along its axis. The number of segments shown (in FIG. 14B, there are eight segments) corresponds with the number of lesions that are to be formed at Lesion Level 1.

In FIG. 14B, all segments of the circle are unmarked. This graphical form indicates at a glance that (i) formation of lesions at this lesion level is now indicated (due to the axial circle view of the lesion level icon), (ii) eight circumferentially spaced lesions are to be formed (due to the number of segments); (iii) no lesions have as yet been formed (by the lack of other markings in the segments).

The location of the Marker 512 also changes to align with Lesion Level 2, with a numeric indicator of 5 mm. This informs the physician that, after Lesion Level 1, the next lesion level to be treated is Lesion Level 2, which is 5 mm below (inferior to) Lesion Level 1.

With the device 26a positioned at Lesion Level 1, the physician actuates the electrodes for a first pre-set period. The balloon icon 516 disappears as treatment progresses on a given level. A Timer Icon 518 shows the application of radio frequency energy for the pre-set period. At the end of this pre-set period (see FIG. 14C), treatment indicia (e.g., dots) appear in four segments of the graphical segmented circle, indicating the formation of the first four lesions, as well as their spatial orientation.

The open segments remaining in the segmented circle prompt the physician to rotate the basket by 45-degrees, and actuate the electrodes for second time. After the pre-set period (tracked by the Timer Icon 518) (see FIG. 14D), more treatment indicia (the dots) appear in the remaining segments of the circle. This indicates that all the lesions prescribed for Lesion Level 1 have been formed, and to deflate the basket and move to the next treatment level. The Marker 512 that is displayed directs the physician to Lesion Level 2, which is 5 mm below Lesion Level 1. The Balloon Icon 516 can reappear to prompt the physician to deflate the balloon.

The physician is thereby prompted to deflate the basket, move to Lesion Level 2, and expand the basket. As FIG. 14E shows, upon sensing electrode impedance, indicating contact with tissue at Lesion Level 2, the UGUI 504 changes the graphical form of Lesion Level 1 back to an edgewise cylinder. The edgewise cylinder for Lesion Level 1 includes an indicator, e.g., checkmark, to indicate that Lesion Level 1 has been treated (as shown in FIG. 14E). The insertion of the treatment completed indicator is yet another graphical form the UGUI 504 displays to communicate status information to the physician.

Also referring to FIG. 14E, upon sensing electrode impedance, indicating contact with tissue at Lesion Level 2, the UGUI 504 changes the graphical form of Lesion Level 2 to the second graphical form, comprising, e.g., the segmented circle, as already described. This is shown for Lesion Level 2 in FIG. 14E. The location of the Marker 512 also changes to align with Lesion Level 3, with a numeric indicator of 5 mm. This informs the physician that after Lesion 2, the next lesion level will be Lesion Level 3, which is 5 mm below (inferior to) Lesion Level 2.

As shown in FIGS. 14F and 14G, with the device 26a positioned at Lesion Level 2, the physician actuates the electrodes for a first pre-set period, then rotates the device 26a 45-degrees, and actuates the electrodes for the second pre-set period. The Timer Icon 518 reflects the application of radio frequency energy for the pre-set periods, and the treatment indicia (e.g., dots) are added to the segments of the graphical segmented circle, indicating the formation of the first four lesions (FIG. 14F) and the next four lesions (FIG. 14G), as well as their spatial orientation.

Upon formation of the eight lesions in Lesion Level 2, the balloon icon 518 again appears. This indicates that all the lesions prescribed for Lesion Level 2 have been formed, and to deflate the basket and move to the next treatment level. The Marker 512 that is displayed directs the physician to Lesion Level 3, which is 5 mm below Lesion Level 2.

The physician is thereby prompted to deflate the basket, move to Lesion Level 3, and expand the basket. Upon sensing electrode impedance, indicating contact with tissue at Lesion Level 3 (see FIG. 14H), the UGUI 504 changes the graphical form of Lesion Level 2 back to an edgewise cylinder (as FIG. 14H shows). The edgewise cylinder for Lesion Level 2 now includes an indicator, e.g., the checkmark, to indicate that Lesion Level 2 has been treated (as FIG. 14H also shows).

As FIG. 14I also shows, upon sensing electrode impedance, indicating contact with tissue at Lesion Level 3, the UGUI 504 changes the graphical form of Lesion Level 3 to the second graphical form, comprising, e.g., the segmented circle, as already described. This is shown for Lesion Level 3 in FIG. 14H. The location of the Marker 512 also changes to align with Lesion Level 4, with a numeric indicator of 5 mm. This informs the physician that after Lesion 3, the next lesion level will be Lesion Level 4, which is 5 mm below (inferior to) Lesion Level 3.

The physician proceeds to form eight lesions in Lesion Level 3 (FIGS. 14I and 14J), then moving on to Lesion Level 4 (not shown, but following the same progression as already described). All the while, the UGUI 504 visually records and confirms progress. As shown in FIG. 14K, the graphical Lesion Level cylinders for Lesion Levels 3 and 4 return edgewise when the desired number of lesions has been formed on the respective level and treatment at the level has been completed. At that time, a check mark appears on the edgewise cylinder, indicating that treatment at that level has been completed for Lesion Levels 1, 2, 3, and 4 (as shown in FIG. 14K).

In FIGS. 14K to 14N, on Lesion Levels 5 and 6, the segments in the segmented circle number twelve, indicating that twelve lesions are to be formed on these levels. In the Levels 5 and 6, there are twelve lesions circumferentially spaced 30-degrees apart (i.e., a first application of energy, followed by a 30-degree rotation of the basket 56, followed by a second application of energy, followed by a 30-degree rotation of the basket 56, followed by a third application of energy). In Level 5, the balloon structure is only partially expanded, whereas in Level 6, the balloon structure 72 is more fully expanded, to provide lesion patterns that increase in circumference according to the funnel-shaped space available in the funnel of the cardia.

The UGUI 504 reflects completion of the treatment (see FIG. 14O) in all levels 1-6.

Thus, the UGUI 504, by purposeful manipulation of different stylized graphical images, visually prompts the physician step wise to perform a process of forming a pattern of lesions comprising a plurality of axially spaced lesion levels, each lesion level comprising a plurality of circumferential spaced lesions. The UGUI 504 registers the formation of lesions as they are generated in real time, both within and at each circumferentially spaced level. The UGUI 504 therefore displays for the physician a visual record of the progress of the process from start to finish. The UGUI 504 assures that individual lesions desired within a given level are not skipped, or that a given level of lesions is not skipped.

In the UGUI 508, each Lesion Level 1 to 6 is initially depicted by a first stylized graphical image comprising an edgewise cylinder with a number identification of its level. When the formation of lesions at a given level is indicated, the UGUI 504 changes the first stylized graphical image into a second stylized graphical image, different than the first image, comprising an axial view of the cylinder, presented as a segmented circle, with the numbers of segments corresponding to the number of lesions to be formed. There also appears juxtaposed with the next lesion level to be treated (still displayed as an edgewise cylinder), a marker along with a number indicating its distance from the present legion level. As the physician manipulates the device 26a to form lesions on the indicated levels, the second graphical image further changes to a third graphical image, different than the first or second images, by adding indicia within the segmented circle to reflect the formation of lesions, to guide the physician to successively rotate and operate the device 26a at the lesion level. Upon forming the desired lesion pattern on a given level, the UGUI 504 again changes the third graphical image to a fourth graphical image, different than the first, second, and third graphical images, comprising an edgewise cylinder with a number identification of its level, and further an indicator (e.g. a check mark) that indicates all desired lesions have been formed at the respective level. A Marker 512 is successively updated to direct the physician to the next Lesion Level. In this way, the UGUI 504 prompts the formation of eight lesions circumferentially spaced 45-degrees apart in the Levels 1, 2, 3, and 4, and the formation of twelve lesions circumferentially spaced 30-degrees apart at Lesion Levels 5 and 6.

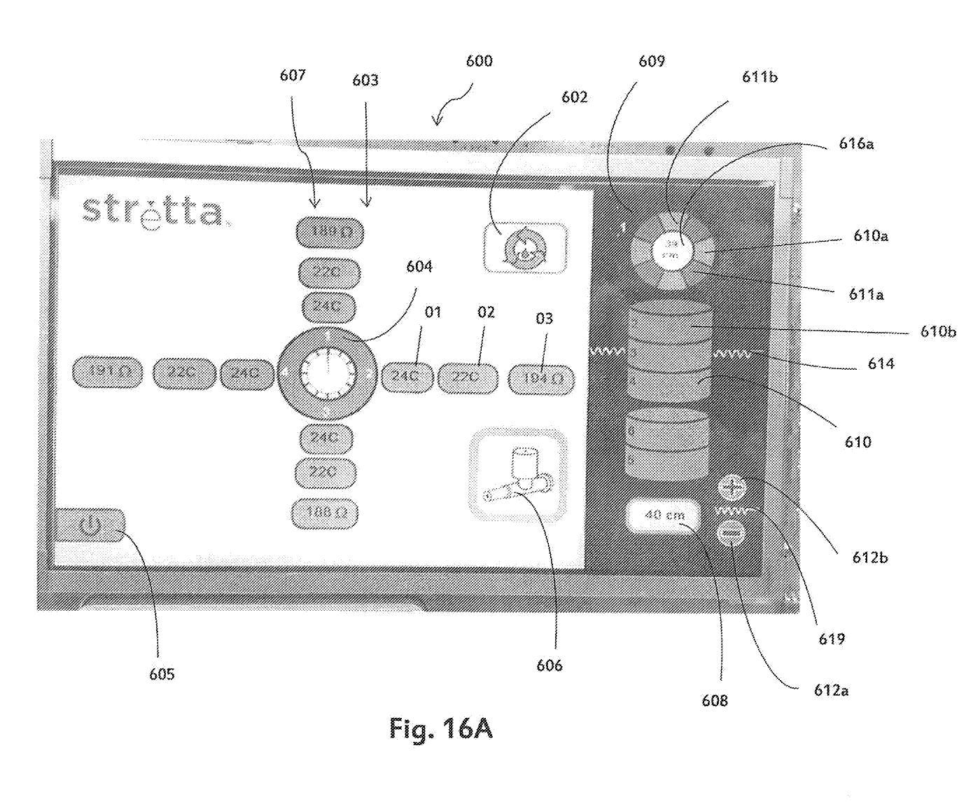

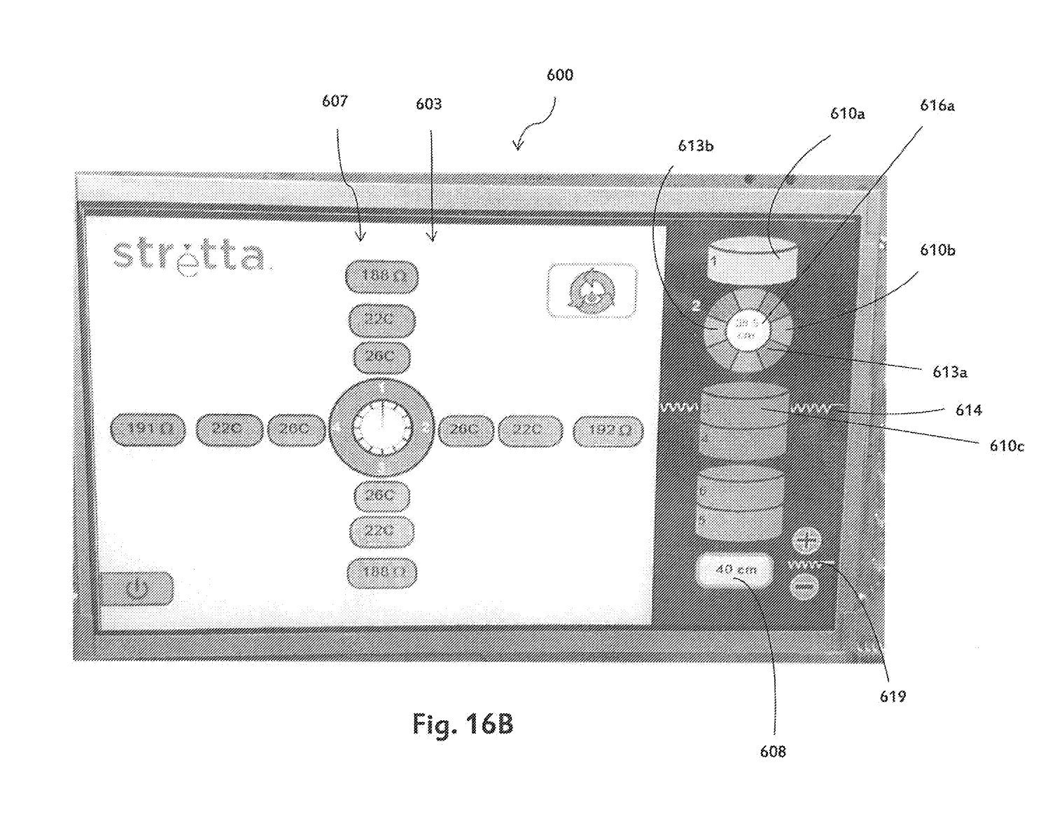

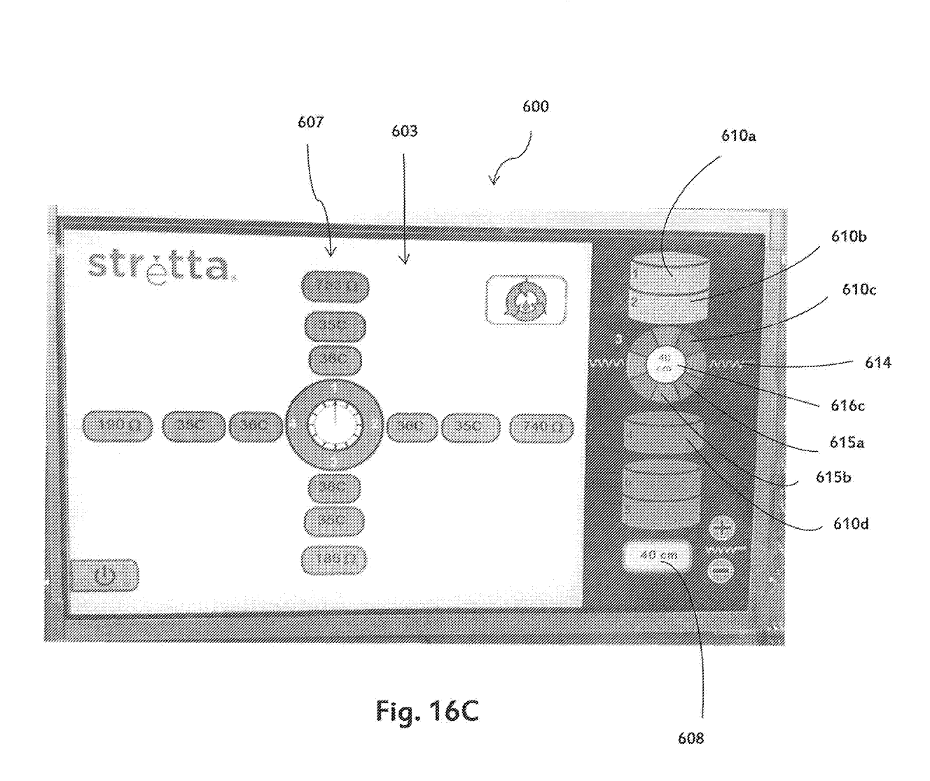

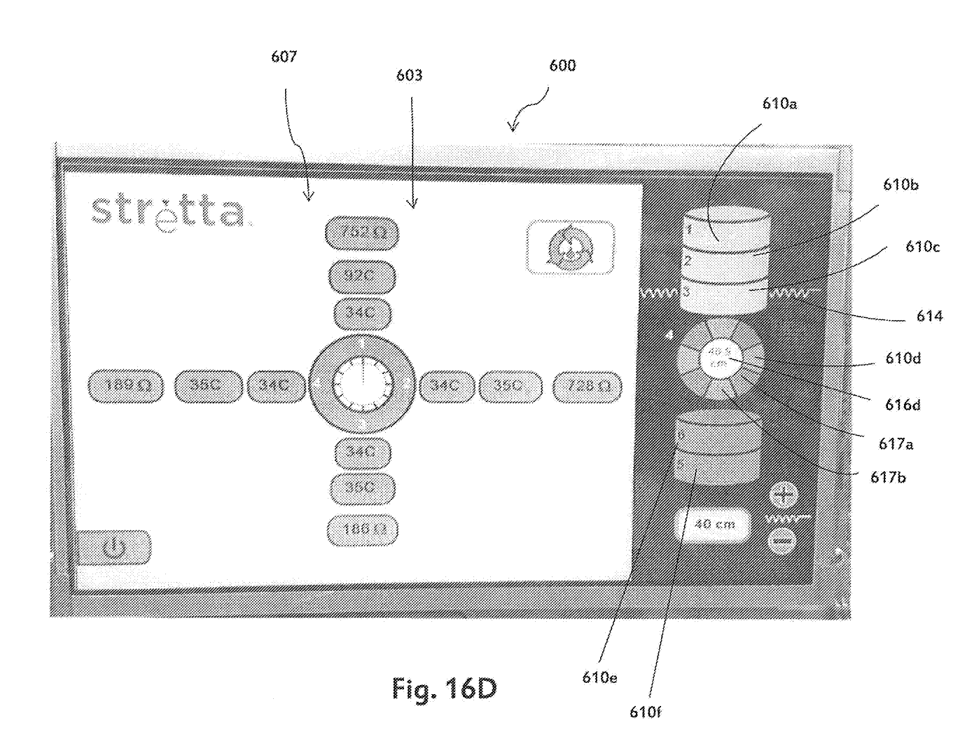

In the alternate embodiment of FIGS. 16A-16D, the system is similar to that of FIGS. 14A-14O, except that a numeric indication is provided to facilitate conveyance of information regarding the positioning of the electrosurgical device relative to the Z-line (or other desired anatomic landmark). It also differs in a few other respects such as the positioning and markings of the lesion level indicators which are discussed below.

Currently, in order to locate the anatomic reference of the Z-line within a patient, an endoscope having measurement markings is inserted transorally through a bite block. The distance from the bite block to the Z-line is then determined (measured) by noting the external marker on the endoscope. A guidewire is then inserted through the endoscope and advanced to the target tissue, and then the endoscope is removed. The clinician either memorizes the measured distance to the Z-line or writes down the measured distance. The electrosurgical device, e.g., device 26a described above, is inserted over the guidewire to the Z-line based on the memorized or written measurement since there is no direct visualization during insertion of device 26a. Thus, since the clinician has measured the distance to the Z-line using the endoscope, the clinician needs to either remember the measurement or remember to write down the measurement, since it is necessary for electrosurgical device placement.

In a preferred embodiment, the first tissue treatment level is a first distance from (above) the Z-line, the second treatment level is a shorter distance than the first distance above the Z-line, the third treatment level is at the Z-line and the fourth treatment level is a distance below the Z-line. At each of these axially spaced levels of tissue, electrosurgical energy is applied by the device 26a as discussed above to form the lesions. If the clinician fails to accurately remember or record the measured distance to the Z-line, the treatment will commence at an improper distance with respect to the Z-line, followed by treatments at spaced intervals from the initial incorrect starting point. The system of the present invention provides a graphic display to minimize the risk of this occurring.

In the embodiment of FIGS. 16A-16D, the graphic display (UGUI) 600 has a treatment screen 603 with an electrode icon 607 which like electrode icon 466 of FIG. 14A spatially models the particular electrode geometry of the treatment device 26a. As shown, four electrodes are shown in the graphic image spaced apart by 90 degrees to reflect the geometry of the four-electrode configuration of device 26a of FIG. 4. The parameter icons have graphic regions O1, O2 and O3 (like in FIG. 14A) to numerically display, respectively, the mucosal temperature, the electrode tip temperature and the derived impedance value for that electrode. The screen 603 provides one example of sensed temperature and impedance for ease of understanding. Clearly such parameters are dependent on the particular use of the treatment device 26a. As shown, the array of parameter icons extend linearly from the timer icon 604 which shows the elapsed time of application of electrosurgical energy by the four electrodes. Note the electrode icon 607 is identical to the icon 466 of FIG. 14A, reflecting the multiple electrode geometry of the treatment device 26a, so that discussion of this icon and the temperature and impedance displays are not further discussed since the above discussion is fully applicable to the electrode icon 607 of FIG. 16A-16D.

Mode switch button 605 provides an indication of the operation mode, e.g., a color indication wherein there is a different color for the setup mode, ready mode and treatment mode. Valve icon 606 appears before the first treatment is commenced when moved from the setup mode to the ready mode to remind the clinician to use the pressure relief valve when the balloon is inflated. The relief valve icon 606 in a preferred embodiment disappears as soon as the first treatment cycle begins. Icon 602 is a fluid flow icon to indicate to the user that fluid flow is occurring. In this embodiment, rather than the user adjusting the fluid flow to different rates, e.g., low, medium, high and maximum, the flow rate is automatically controlled. Therefore, unlike the screen of FIG. 14A which depicts the pump speed, the fluid flow icon 602 appears to show a fluid animation when fluid flow is occurring, representative of any of the speeds automatically selected by the system.

Note that the automatic pump adjustment advantageously strikes a balance between providing a sufficient amount of cooling fluid to cool tissue while minimizing the risk of aspiration pneumonia if too much fluid is applied and water gets into the lungs. This is achieved with the system's software as fluid flow starts low by default and as the tissue temperature climbs, and reaches a certain threshold for example, the system will automatically increase the flow. The benefit of this can be appreciated since if left up to the clinician, the clinician could start fluid flow at a maximum rate which could cause too much fluid and risk of pneumonia. With this automatic system, conversely if the temperature is lower, the flow rate will adjust downwardly. The automatic system in certain embodiments helps to ensure non-ablation of tissue by automatically providing a sufficient cooling of tissue.

With further reference to the treatment screen 603 of UGUI 600, the treatment screen 603 also includes another region adjacent the region containing the electrode icon 607 which depicts the plurality of axially spaced levels of tissue (also referred to as tissue levels), i.e., spatially models the desired lesion levels and the number of lesions in each level. As in the embodiment of FIGS. 14A-14O, the lesion level icons change in real time to step-wise guide the physician through the procedure and to record the progress of the procedure from start to finish as the number of lesions formed and unformed are depicted. As in the embodiment of FIG. 14A, each lesion level icon 610a, 610b, 610c, 610d, 610e, 610f (collectively lesion level icons 610) has a number 1-6 associated with it to represent the respective lesion level. Also, as in FIG. 14A, the lesions level icons 610 have the geometric shape of a disc or cylinder with the lesion level numeric indicator shown within the disk, and like in FIGS. 14A-14O, the disc is removed from the stacked array when that lesion level is being treated and the numeric indicator is moved adjacent the disk. The Z-line is represented by squiggle line 614.

However, whereas the UGUI of FIG. 14A depicts for each lesion level icon a distance from the previous lesion level, the UGUI 600 of FIG. 16A numerically indicates the actual distance of the electrodes from the reference point. That is, the lesion level icon 610 has a number associated with it that provides the distance from the fixed reference point which is determined from the fixed point outside the patient, e.g., a fixed point from an external bite block, to the targeted region of the patient's body. In the illustrated embodiment, this numeric distance is shown within the disc, although alternatively it could be placed adjacent the disc. As each lesion level is treated, the numeric indicator of the distance from the fixed reference changes to reflect the distance of the new lesion level. In a preferred embodiment, the distance appears in the disc when the lesion level is being treated and does not appear in the other discs, i.e., the discs of the non-selected lesion levels.

The measured distance X from the fixed reference to the Z-line is inputted by the clinician. More specifically, there is a predetermined (preset) value Y stored in the controller which initially appears (from a default blank state) in window 608 if the clinician desires to initiate the measurement/distance system. In one embodiment, the preset value Y is 38.0 cm, although other preset values are also contemplated. Consequently, if the clinician wishes to enter the measured distance X, the clinician presses the + (plus) or − (minus) button, 612b, 612a, respectively, to initiate the measurement system. After pressing either button 612a, 612b, the preset value Y will appear in window 608. The clinician then presses the + or − button 612b, 612a, (collectively the “adjustment buttons 612”) to adjust the Y value either up (+) or down (−) until the number in the window equals X. (The clinician need not press the + or − button 612b, 612a to adjust the value if Y initially equals X). In one embodiment, each press of the adjustment button 612 changes the value in window 608 in 0.5 centimeter increments, e.g., a first press of the + button 612b adjusts the number in window 608 to Y+0.5 and a first press of the − button 612a adjusts the number in the window 608 by Y−0.5 cm. This adjustment by pressing the respective button 612a, 612b occurs until the number X is reached. Subsequently, the clinician can commence treatment.

Note in a preferred embodiment, the adjustment buttons 612 are disabled during the treatment cycle, i.e., when the radiofrequency energy is being applied to the tissue level to form lesions. However, preferably, the adjustment buttons 612 are enabled between treatments to enable the clinician to go back in and re-measure and re-input the distance to the Z-line if the patient moves and the position of the Z-line changes and the distance needs to be re-calculated.

The lesion level icons 610 numeric distance indicators are responsive to the distance input, wherein in the preferred embodiment, the first lesion level icon 610a indicating X−1 cm, the second lesion level icon 610b indicating X−0.5 cm, the third lesion level icon 610c indicating X and the fourth lesion level icon 610d indicating X+0.5 cm.

Turning now to FIGS. 16A-16D which show an example of the changing screen 603 wherein a distance from the fixed reference point outside the patient to the designated region inside the patient, i.e., the Z-line, has been measured and inputted at 40 cm (X=40 cm). Note screen 603 of FIG. 16A is representative after the value X has been inputted after adjustment of the Y value. That is, in this embodiment, if a preset value was 38.0 mm, the clinician would have pressed either button 612a, 612b to initiate the measurement system and then pressed the + button 612b four times so that the value in the window 608 reached 40 cm. Note 40 cm is used by way of example since different measurements would yield different results and different input and adjustments. Additionally, preset values other than 38.0 cm are also contemplated.

With this distance of 40 cm inputted, when the electrodes are placed in desired contact with the targeted tissue region in lesion level 1, the first treatment level is automatically indicated at 39 cm (X−1 mm) as shown in region 616a of the now axially facing first lesion level icon 610a. The lesions are then formed (in the manner described above) as indicated in each of the eight quadrants (segments), with the quadrants of the segmented circle color coded to indicate lesion formation. In some embodiments, a blue colored quadrant indicates the region not yet treated and a green colored quadrant indicates the target site selected to be treated, i.e. target site for formation of lesions. In the black and white Figures of this patent application, the green regions 611b (and 613b, 615b and 617b) being treated are shown as light colors and the non-treated blue color regions 611a (and 613a, 615a and 617a) are shown as darker colors. Note that other colors or other indicators to mark treated and untreated regions in the lesion level icons 610 of screen 603 are also contemplated. Note that during treatment the lesion level numeric indicator appears adjacent rather than inside the lesion level icon 610. After treatment, the region is indicated in color gray, which in the black and white Figures of this application is shown as a lighter color.

After completion of treatment at lesion level 1, lesion level icon 610a is rotated back to its original edgewise facing position above the remaining lesion level icons 610c-610f of the stacked array and the next lesion level icon 610b, corresponding to lesion level 2, is removed from the array and rotated from its edgewise facing to an axial circle view to show the distance 39.5 cm in the central region 616b of lesion level icon 610b (FIG. 16B) as the device 26a is moved to the next level. Note, that after completion of treatment at lesion level 1 and rotation of lesion level icon 610a back to its edgewise orientation, a color coding, such as a gray color, can be utilized to indicate completion of treatment at the lesion level. Note in the black and white Figures of this application, the lesion level icon after treatment is shown as a lighter color that the stacked lesion level icons not yet treated.

As with lesion level 1, FIG. 16B illustrates some lesions formed (quadrants 613b) and others not yet formed (quadrants 613a) in lesion level 2. After completion of treatment at lesion level 2 (rotation of the device and application of energy), lesion level icon 610b is returned to the stacked array (facing edgewise) underneath lesion level icon 610a (FIG. 16B), its color changed (e.g., changed to gray), lesion level icon 610c (for lesion level 3) is removed from the array and rotated (axially facing) to show the distance 40 centimeters (cm) (FIG. 16C) which is at the Z-line. Note in some embodiments energy is applied for a set time, e.g., a 60 second cycle, then the screen freezes for a short period of time, e.g., 8 seconds, wherein the lesion level icon returns to its sidewise (edgewise) position and the next lesion level icon is rotated. This refreshing/changing of the screen after a timed cycle is applicable to each of the lesion level icons. Note different time periods are also contemplated. Note that an adjustment, e.g., up/down, button can be provided on the screen to enable the clinician to return to a lesion level or skip a lesion going backward (down) or forward (up), with the corresponding numeric distance value appearing with the selected lesion level.

FIG. 16C illustrates some lesions formed (quadrants 615b) and others not yet formed (quadrants 615a). After completion of treatment at lesion level 3, lesion level icon 610c is returned to the stacked array (facing edgewise) under lesion level icon 610b, its color changed, and the next lesion level icon 610d representing lesion level 4, is removed from the array and rotated to show the distance 40.5 cm (FIG. 16D) which is 0.5 cm below the Z-line. FIG. 16D illustrates some lesions formed (quadrants 617b) and others not yet formed (quadrants 617a). Next, after completion of treatment at lesion level 4, lesion level 4 is returned to the stacked array under lesion level icon 610c and its color changed.

For treatment levels 5 and 6, instead of valve icon 606, a balloon icon appears (not shown) to indicate for treatment level 5 inflation to a pre-set level, e.g., a fully inflated level of 25 ml, and for treatment level 6 a balloon icon appears (not shown) to indicate to deflate the balloon to a pre-set level, e.g., a partially inflated level of 22 ml.

As can be appreciated, the lesion level icons 610 depict the incremental changes in distances, e.g., 0.5 cm increments, with respect to the selected targeted region in the body, showing the distances from the fixed reference point which are initially above the Z-line then at the Z-line then below the Z-line, all indicated by reference to the actual measured distance once the distance to the Z-line has been determined and entered by the clinician.

Note that FIGS. 16A-16D illustrate the lesion levels partially treated, e.g., half the lesions formed and half the lesions not yet formed. This corresponds to the condition of formation of lesions by the electrodes in the first pre-set period but prior to formation of lesions in the second pre-set period after 45 degree rotation of device 26a as described above. Clearly if all lesions are formed all the quadrants would be so colored. Also, after completion of treatment at the selected lesion level, as noted above, the color of the lesion level icon changes and it is rotated to its edgewise position above the subsequent lesion level icon to be treated and stacked under the preceding lesion level icon already treated. Alternately, it can change color after rotated back to its edgewise position after treatment. As can be appreciated, the status of treatment of all the lesion levels can be viewed and assessed simultaneously on the single screen 603 to aid the clinician in assessing the progress of the surgical procedure.

Note that FIGS. 16A-16D differ from FIGS. 14a-14O in the display of actual distances as described in detail above. FIGS. 16A-16D also differ from FIGS. 14A-14O in that the removed and rotated lesion level icon appears within the stacked array sequence rather than below the stacked array, with the lesion level icon representing the lesion level 1-4 being treated positioned between the lesion level icons representing the treated levels and not yet treated levels. Additionally, in FIGS. 16A-16D, the markings for formation of lesions are color coded fillings within the quadrants of the disc. Also note, lesion level icons for levels 5 and 6 are grouped slightly spaced from levels 1-4 and are also numbered in order of treatment so that level 5 is below level 6.

2. The LGUI

The LGUI 506 (FIG. 15A) generates a graphical user display that guides the physician in manipulating the device 26b to form a prescribed lesion pattern in the anal canal, as shown in FIG. 9. The lesion pattern comprises a plurality of axially spaced lesion levels (in the illustrated embodiment, numbered 1 to 5), each lesion level comprising a plurality of circumferential spaced lesions (in the illustrated embodiment, there are sixteen lesions, arranged in sets of four).

The display of the LGUI 506 (see FIG. 15A) shows Lesion Levels 1, 2, 3, 4, and 5, corresponding with the multiple lesion levels to be formed in the anal canal. Lesion Levels 1 to 5 are displayed as segmented discs, numbered 1 to 5, which are tilted slightly on their axes, and arranged one above the other. Each disc is divided into four quadrants.

The LGUI 506 also shows (see FIG. 15A) a dentate squiggle line 514. In preparation for the treatment, the physician visualizes in the anal canal the dentate line or other desired anatomic landmark. Markers are arranged at 5 mm intervals along the barrel of the device 26b. Upon visualizing the dentate line, the physician notes the external marker on the barrel that corresponds to this position. With reference to the markers, the physician can then axially advance or retract the barrel in 5 mm increments, which correspond to the spacing between the lesion levels.

Next to the graphical form of the disc of Lesion Level 1 is a Guide Marker 512 (see FIG. 15A). The Guide Marker 512 indicates that formation of lesions in Lesion Level 1 is indicated. A numeric value (5 mm) is displayed in association with the edgewise cylinder of Lesion Level 1, which indicates that Lesion Level 1 is 5 mm from the anatomic landmark.

In FIG. 15A, all quadrants of the lesion level discs are unmarked. This graphical form indicates at a glance that (i) formation of lesions at Lesion Level 1 is now indicated (due to the position of the Marker 512) and (ii) no lesions have as yet been formed (by the lack of markings in the quadrants).

The device 26b includes an array of four needle electrodes arrange in an arc, which can be advanced and retracted (see FIG. 6). The array of needle electrodes is positioned at Level 1, in alignment with quadrant 1, and advanced. The physician actuates the electrodes for a first pre-set period. A Timer Icon 518 shows the application of radio frequency energy for the pre-set period. At the end of this pre-set period, treatment indicia (e.g., four dots) appear in the first quadrant of the graphical segmented discs (see FIG. 15B), indicating the formation of the first four lesions, as well as their spatial orientation in the first quadrant.

The location of the Marker 512 also changes to align with Lesion Level 2, with a numeric indicator of 5 mm. This informs the physician that after Lesion Level 1, the next lesion level will be Lesion Level 2, which is 5 mm above (superior to) Lesion Level 1.

Upon the satisfactory creation of the lesion pattern in the first quadrant of Level 1, as just described, and as prompted by the Marker 512 (now aligned with Lesion Level 2), the physician actuates the button to move the needle electrodes back to their retracted positions. Still grasping the hand grip and visualizing through the viewing port, the physician moves the barrel 5 mm axially upward to Level 2, remaining rotationally aligned in the first quadrant. The physician again deploys the needle electrodes and performs another lesion generating sequence. The location of the Marker 512 changes to align with Lesion Level 3, with a numeric indicator of 5 mm. This informs the physician that after Lesion Level 2, the next lesion level will be Lesion Level 3, which is 5 mm above (superior to) Lesion Level 2. Treatment indicia (e.g., four dots) appear in the first quadrant of the graphical segmented disc of Lesion Level 2 (see FIG. 15C), indicating the formation of the four lesions, as well as their spatial orientation in the first quadrant.

The physician repeats this sequence of steps until additional number of lesion patterns are formed within the axially spaced first quadrants in Levels 2, 3, 4, and 5 (see FIGS. 15D, 15E, and 15F). The location of the Marker 512 also changes to align with successive Lesion Levels, to guide the physician through the lesion levels. Treatment indicia (e.g., four dots) appear in the first quadrant of the graphical segmented discs of Lesion Levels 2, 3, 4, and 5 (see FIG. 15F), indicating the formation of the four lesions, as well as their spatial orientation in the first quadrant.

Upon formation of the four lesions in quadrant 1 of Lesion Level 5, the Marker 512 returns to Lesion Level 1 (see FIG. 15F), prompting the physician to return to Lesion Level 1, and rotate the barrel a selected arcuate distance at Lesion Level 1 into alignment with the second quadrant, i.e., by rotating the barrel by ninety degrees.

Guided by the LGUI 506, the physician again deploys the needle electrodes and performs another lesion generating sequence at quadrant 2 of Level 1. Guided by the LGUI 506 (as shown in FIG. 15G), and following the Marker 512, the physician then moves the barrel axially upward in 5 mm increments, sequentially to quadrant 2 of Lesion Level 2, then quadrant 2 of Lesion Level 3, then quadrant 2 of Lesion Level 4, and quadrant 2 of Lesion Level 5. At each Lesion Level, the physician deploys the needle electrodes and performs another lesion generating sequence at quadrant 2 of the respective level. After lesion formation at each Lesion Level, treatment indicia (e.g., four dots) appear in the second quadrant of the graphical segmented discs of Lesion Levels 2, 3, 4, and 5 (see FIG. 15G), indicating the formation of the four lesions, as well as their spatial orientation in the second quadrant.

Upon formation of the four lesions in quadrant 2 of Lesion Level 5, the Marker 512 returns to Lesion Level 1. The physician returns to Lesion Level 1, and again rotates the barrel a selected arcuate distance at Lesion Level 1 into alignment with the third quadrant, i.e., by rotating the barrel by ninety degrees.

Guided by the LGUI 506 (see FIG. 15H), the physician again deploys the needle electrodes 48 and performs another lesion generating sequence at quadrant 3 of Level 1. Treatment indicia (e.g., four dots) appear in the quadrant 3 of the graphical segmented disc of Lesion Level 1, indicating the formation of the four lesions, as well as their spatial orientation in the third quadrant.

As shown in FIG. 15H, guided by the LGUI 506, and following the Marker 512 as it advances with lesion formation at each level, the physician then moves the barrel axially upward in 5 mm increments, sequentially to quadrant 3 of Lesion Level 2, then quadrant 3 of Lesion Level 3, then quadrant 3 of Lesion Level 4, and quadrant 3 of Lesion Level 5. At each Lesion Level, the physician deploys the needle electrodes and performs another lesion generating sequence at quadrant 3 of the respective level. Treatment indicia (e.g., four dots) appear in the third quadrant of the graphical segmented discs of Lesion Levels 2, 3, 4, and 5 (see FIG. 15H), indicating the formation of the four lesions, as well as their spatial orientation in the third quadrant.

The physician repeats the above described sequence one additional time, returning the barrel to Lesion Level 1 and again rotate the barrel e.g., ninety degrees, into alignment with quadrant 4 of Lesion Level 1 (see FIG. 15I). The physician forms the lesion patterns in quadrant 4 in the Levels 1, 2, 3, 4, and 5. Treatment indicia (e.g., four dots) appear in the fourth quadrant of the graphical segmented discs of Lesion Levels 1, 2, 3, 4, and 5 (see FIG. 15I), indicating the formation of the four lesions, as well as their spatial orientation in the fourth quadrant. In addition, with the formation of lesions in the fourth quadrant at each Lesion Level, i.e., each quadrant marked by four dots (indicating completion of lesion creation) an indicator, e.g., a checkmark, appears adjacent the disc to indicate that the respective Lesion Level has been treated (see FIG. 15I) which shows completion of all Levels 1-5 and therefore a checkmark next to each level.

As described, the LGUI 506 visually prompts a user in a step-wise fashion to perform a process of forming a pattern of lesions in the anal canal comprising a plurality of axially spaced lesion levels, each lesion level comprising a plurality of circumferential spaced lesions. The LGUI 506 registers the formation of lesions as they are generated in real time, both within and at each circumferentially spaced level. The LGUI 506 displays for the user a visual record of the progress of the process from start to finish and guides the user so that individual lesions desired within a given level are all formed, and that a given level of lesions is not skipped.

Each Lesion Level 1 to 5 of the LGUI 506 is depicted by a first stylized graphical image comprising an edge-tilted disc with a number identification of its level. The discs are segmented corresponding to the regions in which lesions are to be formed. There also appears juxtaposed with the next lesion level to be treated, a marker along with a number indicating its distance from the present legion level. As the physician manipulates the device 26b to form lesions on the indicated levels, the graphical image further changes to a second graphical image, different than the first image, by adding indicia within the segmented circle to reflect the formation of lesions, to guide the physician as the device is successively operated at the lesion level. The graphical images continue to change to reflect formation in each quadrant in each Lesion Level. Upon forming the desired lesion pattern within all quadrants of the Lesion Level, the LGUI 506 again changes to a different graphical image, comprising an indicator (e.g. a check mark) indicating that all desired lesions have been formed at the level.

Note, during the procedure the Marker 512 is updated to direct the physician to the next Lesion Level. In this way, the LGUI 506 prompts the formation of four lesions in sets of four (totaling twelve lesions) circumferentially spaced apart in the Levels 1, 2, 3, 4, and 5.

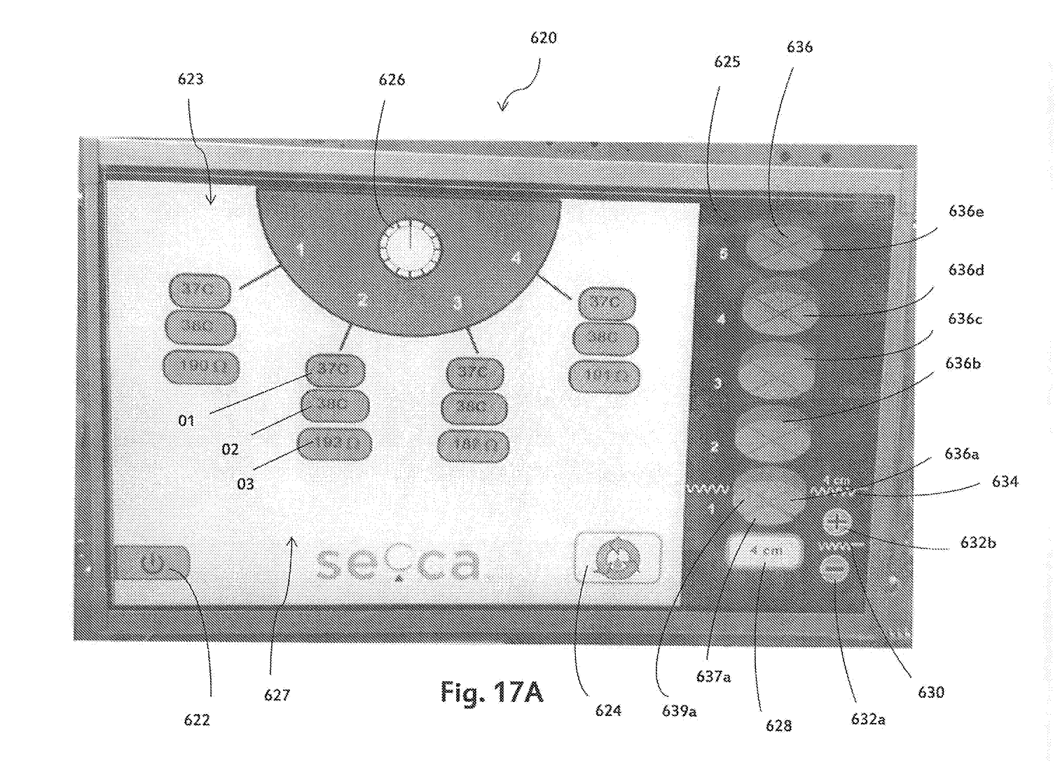

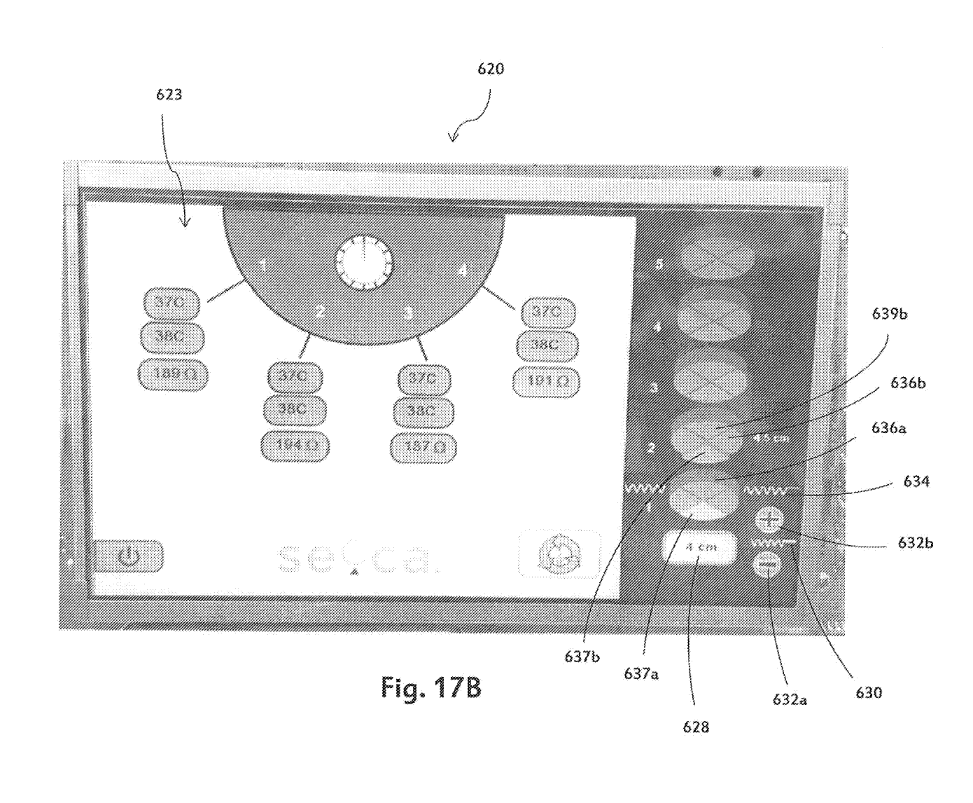

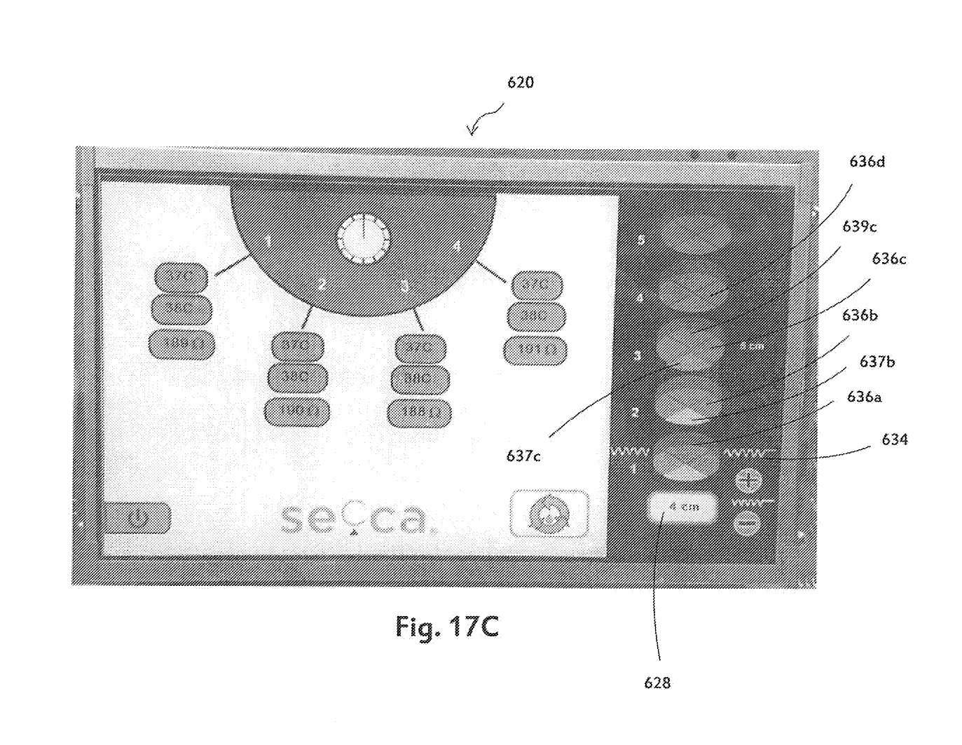

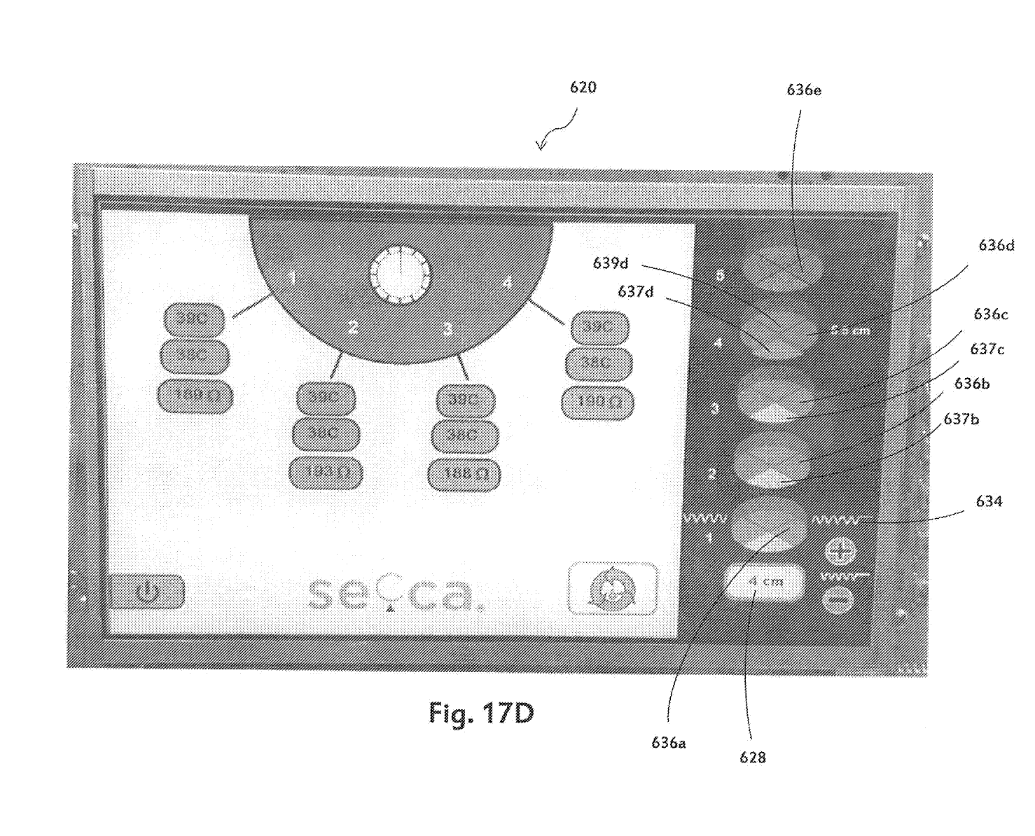

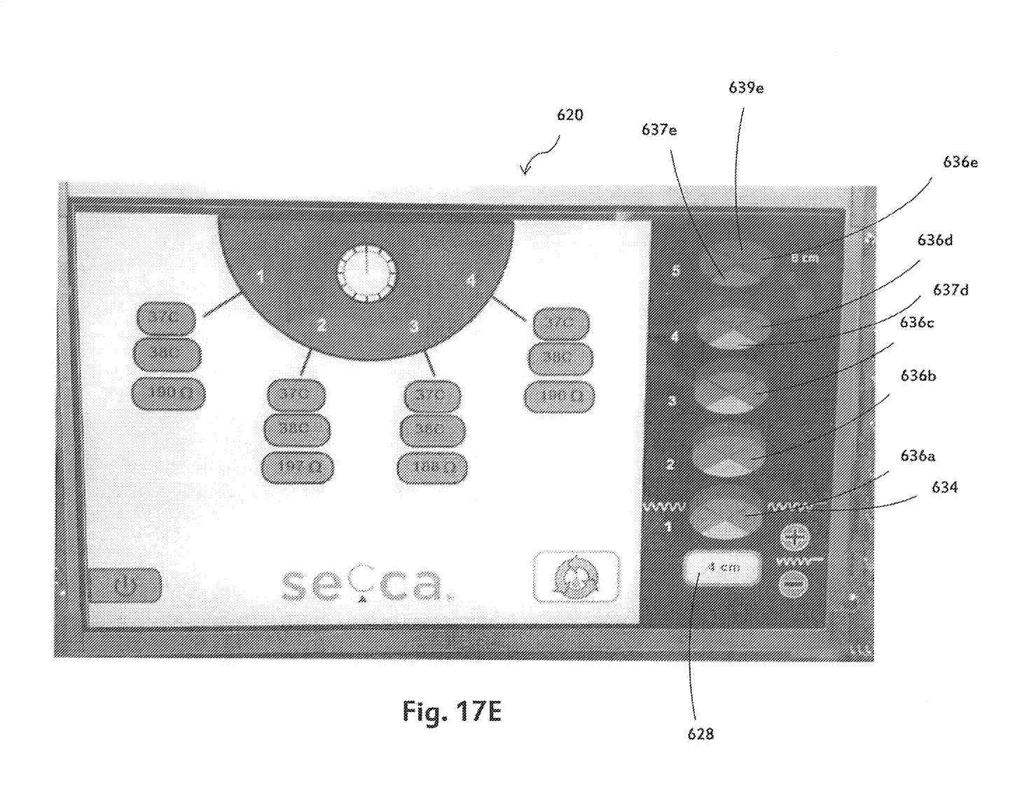

In the alternate embodiment of FIGS. 17A-17E, the system is similar to that of FIGS. 15A-15I, except that a numeric indication is provided to facilitate conveyance of information regarding the positioning of the electrosurgical device relative to the dentate line (or other desired anatomic landmark). It also differs in a few other respects which are discussed below.

Currently, in order to locate the anatomic reference of the dentate line within a patient, the user visualizes the anatomy through the barrel of the device such as device 26b of FIG. 6 which has external measurement markings. The distance from the external reference point (marking) is determined (measured) by noting the external marker number from a series of markers on the device. The clinician either memorizes the measured distance from the anal verge (opening of the anus) to the dentate line or writes down the measured distance. The electrosurgical device, e.g., device 26b described above, is inserted to the dentate line based on the memorized or written measurement. Thus, the clinician needs to either remember the measurement or remember to write down the measurement, since it is necessary for electrosurgical device placement.

In a preferred embodiment, the first tissue treatment level is at the dentate line, the second treatment level is a first distance from (above) the dentate line, the third treatment level is at a second distance from (above) the dentate line which is greater than the first distance, the fourth treatment level is at a third distance from (above) the dentate line which is greater than the second distance, and the fifth treatment level is at a fourth distance from (above) the dentate line which is greater than the third distance. At each of these axially spaced levels of tissue, electrosurgical energy is applied by the device 26b as discussed above to form the lesions. If the clinician fails to accurately remember or record the measured distance to the dentate line, the treatment will commence at an improper distance with respect to the dentate line, followed by treatments at spaced intervals from the initial incorrect starting point. The system of the present invention provides a graphic display to minimize the risk of this occurring.

In the embodiment of FIGS. 17A-17E, the graphic display (LGUI) 620 has a treatment screen 623 with an electrode icon 627 which like electrode icon 466 of FIG. 15A spatially models the particular electrode geometry of the treatment device 26b positioned in the anal canal. As shown, four electrodes are shown in the graphic image spaced apart along an arc to reflect the geometry of the four-electrode configuration of device 26b of FIG. 6. The parameter icons have graphic regions O1, O2 and O3 like in FIG. 15A to numerically display, respectively, the mucosal layer temperature, the electrode tip temperature and the derived impedance value for that electrode. The screen 623 provides one example of sensed temperature and impedance for ease of understanding. Clearly such parameters are dependent on the particular use of the treatment device 26b. As shown, the array of parameter icons extends radially in relation to the timer icon 626 which shows the elapsed time of application of electrosurgical energy by the four electrodes. Note the electrode icon 627 is identical to the icon 466 of FIG. 15A, reflecting the multiple electrode geometry of the treatment device 26b, so that discussion of this icon and the temperature and impedance displays are not further discussed since the above discussion is fully applicable to the electrode icon 627 of FIGS. 17A-17E.

Mode switch button 622 provides an indication of the operation mode, e.g., a color indication wherein there is a different color for the setup mode, ready mode and treatment mode. Icon 624 is a fluid flow icon to indicate to the user that fluid flow is occurring. In this embodiment, rather than the user adjusting the fluid flow to different rates, e.g., low, medium, high and maximum, the flow rate is automatically controlled. Therefore, unlike the screen of FIG. 15A which depicts the pump speed, the fluid flow icon 624 appears when fluid flow is occurring, representative of any of the speeds automatically selected by the system.

Treatment screen 623 of LGUI 620 also includes another region adjacent the region containing the electrode icon 627 which depicts the plurality of axially spaced levels of tissue (also referred to as tissue levels), i.e., spatially models the desired lesion levels and the number of lesions or regions for lesion formation in each level. As in the embodiment of FIGS. 15A-15I, the lesion level icons change in real time to step-wise guide the physician through the procedure and to record the progress of the procedure from start to finish as the number of lesions formed and unformed are depicted. As in the embodiment of FIG. 15A, each lesion icon 636a, 636b, 636c, 636d, 636e (collectively lesion level icons 636) has a number 1-5 associated with it to represent the respective lesion level. Also, as in FIG. 15A, the lesion level icons 636 have the geometric shape of a disc or cylinder tilted slightly on their axes, arranged in an array one above the other and divided (segmented) into four quadrants representing anterior, left, posterior and right. A dentate squiggle line 634 is shown adjacent lesion level icon 636a.

However, whereas the LGUI of FIG. 15A depicts for the lesion level icon a distance from the previous lesion level, the LGUI 620 of FIG. 17A numerically indicates the actual distance of the electrodes from the fixed reference point, i.e. the anal verge. That is, the lesion level icon 636 has a number associated with it that provides the distance from the fixed reference point which is determined from the fixed point at the anal opening of the patient, e.g., the distance from the anal verge to the dentate line (designated region of the patient's body). In the illustrated embodiment, this numeric distance is shown adjacent the disc. As each lesion level is treated, the numeric indicator of the distance from the fixed reference changes to reflect the distance of the device and new lesion level from the reference. In a preferred embodiment, the distance only appears adjacent the disc when the corresponding lesion level is being treated and the distances do not appear adjacent the other discs, i.e., the discs of the non-selected lesion levels. Thus, the numeric indictor automatically changes in response to axial repositioning of the electrodes within the patient.

The measured distance X from the fixed reference to the dentate line is inputted by the clinician. More specifically, there is a predetermined (preset) value Y, which initially appears (from a default blank state) in window 628 if the clinician desires to initiate the measurement/distance system. In one embodiment the preset value Y is 2 cm, although other preset values are also contemplated. Consequently, if the clinician wishes to enter the measured distance X, the clinician presses the + (plus) or − (minus) button, 632b, 632a, respectively, to initiate the measurement system. After pressing either button 632a, 632b, the preset value Y will appear in window 628. The clinician then presses the + or − button 632b, 632a, (collectively the “adjustment buttons 632”) to adjust the Y value either up (+) or down (−) until the number in the window equals X. (The clinician need not press the + or − button 632b, 632a to adjust the value if Y initially equals X). In one embodiment, each press of the adjustment button 632 changes the value in window 628 in 0.5 centimeter (cm) increments, e.g., a first press of the + button adjusting the number in window 628 to Y+0.5 and a first press of the − button adjusting the number in the window 628 by Y−0.5 cm. This adjustment by pressing the respective button 632a, 632b occurs until the number X is reached. Subsequently, the clinician can commence treatment.

Note in a preferred embodiment, the adjustment buttons 632 are disabled during the treatment cycle, i.e., when the radiofrequency energy is being applied to the tissue level to form lesions. However, preferably, the adjustment buttons 632 are enabled between treatments to enable the clinician to go back in and re-measure and re-input the distance to the dentate line.

The lesion level icons 636 numeric distance indicators are responsive to the distance input, wherein in the preferred embodiment, the first lesion level icon 636a indicating X, the second lesion level icon 636b indicating X+0.5 cm, the third lesion level icon 636c indicating X+1 cm, the fourth lesion level icon 636d indicating X+1.5 cm, and the fourth lesion level icon 636e indicating X+2 cm.

Turning now to FIGS. 17A-17D which show an example of the changing screen 623 wherein a distance from the fixed reference point, i.e., the anal verge, to the designated region inside the patient, i.e., the dentate line, has been measured and inputted at 4 cm (X=4 cm). Note screen 623 of FIG. 17A is representative after the value X has been inputted after adjustment to the Y value. That is, in this embodiment, if a preset value was 2 cm, the clinician would have pressed either button 632a, 632b to initiate the measurement system and then pressed the + button 632b four times so that the value in the window 628 reached 4 cm. Note 4 cm is used by way of example since different measurements would yield different results and different input and adjustments. Additionally, preset values other than 2 cm are also contemplated.

With this distance of 4 cm inputted, when the electrodes are placed in desired contact with tissue in lesion level 1, the first treatment level is automatically indicated at 4 cm as shown adjacent the lesion level icon 636a. The lesions are then formed at lesion level 1 in alignment with one of the four quadrants, with the quadrants of the segmented circle color coded to indicate lesion formation. In the preferred embodiment, the quadrant corresponding to the selected segment for treatment is shown slightly removed (spaced) from the other quadrants. In some embodiments, a blue colored quadrant indicates the region not yet treated, a green colored quadrant indicates the target site for treatment (targeted for formation of lesions), and a gray colored quadrant indicates completion of the treatment cycle, i.e., formation of lesions. In the black and white Figures of this patent application, the targeted green quadrants to be treated (labeled in the “637 series”) and the non-treated blue quadrants (labeled in the “639 series”) are not discernible, however, this does not affect understanding since the green color quadrants 637a, 637b, 637c, 637d, 637e are the quadrants slightly removed from the disc. In the black and white Figures of this patent application, the completed gray colored quadrants are shown as a lighter color. Note that other colors or other indictors to mark treated and untreated regions in the lesion level icons 636 of screen 623 are also contemplated. Note that during treatment the lesion level numeric indicator appears adjacent the lesion level icon 636.

After completion of treatment at a first region (quadrant) of lesion level 1, the electrodes are retracted and the device 26b is advanced axially to lesion level 2 wherein quadrant 637b of lesion level icon 636b is slightly removed and lesion level icon 636a corresponding to lesion level 1 is shown with quadrant 637a grayed to indicate formation of lesions (FIG. 17B). The numeric indicator 4.5 cm now appears adjacent the lesion level icon 636b, corresponding to lesion level 2, to indicate positioning of 4.5 cm from the reference point and the quadrant 637b would be colored green to indicate selection for treatment. Note as with the UGUI discussed above, changing of one or more of the lesion icons can be based on timed intervals, e.g., 60 second application of energy, followed by 8 second freeze, followed by movement of quadrant. (Other intervals are also contemplated.)

After completion of treatment at a first region (quadrant) of lesion level 2, the electrodes are retracted and the device 26b is advanced axially to lesion level 3 wherein quadrant 637c of lesion level icon 636c is slightly removed and lesion level icon 636b corresponding to lesion level 2 is shown with quadrant 637b grayed to indicate formation of lesions (FIG. 17C). The numeric indicator 5 cm now appears adjacent the lesion level icon 636c, corresponding to lesion level 3, to indicate positioning of 5 cm from the reference point and the quadrant 637c would be colored green to indicate selection for treatment.

After completion of treatment at a first region (quadrant) of lesion level 3, the electrodes are retracted and the device 26b is advanced axially to lesion level 4 wherein quadrant 637d of lesion level icon 636d is slightly removed and lesion level icon 636c corresponding to lesion level 3 is shown with quadrant 637c grayed to indicate formation of lesions (FIG. 17D). The numeric indicator 5.5 cm now appears adjacent the lesion level icon 636d, corresponding to lesion level 4, to indicate positioning of 5.5 cm from the reference point, and the quadrant 637d would be colored green to indicate selection for treatment.

After completion of treatment at a first region (quadrant) of lesion level 4, the electrodes are retracted and the device 26b is advanced axially to lesion level 5 wherein quadrant 637e of lesion level icon 636e is slightly removed and lesion level icon 636d corresponding to lesion level 4 is shown with quadrant 637d grayed to indicate formation of lesions (FIG. 17E). The numeric indicator 6 cm now appears adjacent the lesion level icon 636e, corresponding to lesion level 5, to indicate positioning of 6 cm from the reference point. Lesions are then formed, indicated in selected green colored quadrant 637e, wherein afterward the quadrant 637e would be marked as gray.

After lesions are formed in each of the axially spaced quadrants in Levels 1-5, the electrodes are retracted and the device 26b is retracted axially and rotated into alignment with the second quadrant of Lesion level 1. Guided by the LGUI, the clinician again deploys the needle electrodes to form lesions within the second quadrant at lesion level 1, corresponding to another quadrant of the lesion level icon 636a. Lesions are then sequentially formed in the second quadrant of the other axially spaced lesion levels 2-5 as described above (and depicted in FIG. 15H) and marked accordingly in lesion level icons 636a-636e. After formation of lesions in the second quadrants of lesion levels 1-5, the electrodes are retracted and the device 26b is again retracted and rotated to a third quadrant of lesion level 1 for formation of lesions at a third quadrant, and then advanced sequentially to all third quadrants of lesion levels 2-5. This device is then retracted and aligned with the fourth quadrant of lesion level 1 and sequentially advanced to the fourth continued quadrants of all lesion levels, which when completed will be indicated by gray colored quadrants in each of the four quadrants of each of the five lesion level icons 636a-636e. In this manner of sequential formation of lesions in corresponding quadrants, the sequence of the embodiment of FIGS. 17A-17D resembles the sequence of FIGS. 15A-15I which shows the process from start to finish.

As can be appreciated, the lesion level icons 636 depict the incremental changes in distances, e.g., 0.5 cm increments, with respect to the selected targeted region in the body, i.e., the dentate line, showing the distances from the fixed reference point which are initially at the dentate line then incrementally above the dentate line, all indicated by reference to the actual measured distance once the distance to the dentate line has been determined and entered by the clinician.

Note that FIGS. 17A-17D illustrate the lesion levels partially treated, e.g., one quarter of the lesions formed and three quarters of the lesions not yet formed. This corresponds to the condition of formation of lesions by the electrodes in the first pre-set period but prior to formation of lesions in the next pre-set period after rotation of device 26a as described above. Clearly, if all lesions are formed then all the quadrants would be so marked/colored. Also, after completion of treatment at the selected lesion level, as noted above, the color of the icon changes and the quadrant returns to its original position to complete the circle. As can be appreciated, the status of treatment of all the lesion levels can be viewed and assessed simultaneously on the single screen 623 to aid the clinician in assessing the progress of the surgical procedure.

Note that as with the UGUI described above, an adjacent, e.g., up/down button can be provided on the screen to return to or skip a lesion level, with the numeric value appearing adjacent the corresponding lesion level icon.

Note that FIGS. 17A-17E differ from FIGS. 15A-15I in the display of actual distances as described in detail above. FIGS. 17A-17E also differ from FIGS. 15A-15I in that the quadrant being treated is slightly removed (separated) from the quadrants of the disc. Additionally, in FIGS. 17A-17E, the markings for formation of lesions are color coded fillings within the quadrants of the disc.

While the above description contains many specifics, those specifics should not be construed as limitations on the scope of the disclosure, but merely as exemplifications of preferred embodiments thereof. Those skilled in the art will envision many other possible variations that are within the scope and spirit of the disclosure as defined by the claims appended hereto.

We claim:

1. A system for visually tracking different axially spaced tissue levels and treatment regions within each axially spaced tissue level for application of radiofrequency energy by electrodes of a device to a body region of a patient to perform a surgical procedure, the system comprising a graphic display configured to display a plurality of tissue level indicators, each tissue level indicator corresponding to one of the different axially spaced tissue levels, and a numeric indicator associated with each tissue level indicator configured to indicate a distance from a fixed reference to a designated region within the patient, the numeric indicator automatically changing in response to axial repositioning of the electrodes of the device within the patient.

2. The system of claim 1, wherein the numeric indicator of a first selected tissue level of the axially spaced tissue levels is configured to disappear when a second tissue level of the axially spaced tissue levels is selected and a different numeric indicator appears.

3. The system of claim 1, wherein the system includes a controller, and the fixed reference is outside the patient and the designated region is the Z-line within a gastro-intestinal tract of the patient, where a measurement made from the fixed reference to the Z-line is inputted to the controller, and a first treatment of the surgical procedure is a first distance from the Z-line and a subsequent second treatment of the surgical procedure is a second distance closer to the Z-line.

4. The system of claim 1, wherein the system includes a controller, and the designated region is a dentate line within the patient, wherein a measurement made from the fixed reference to the dentate line is inputted to the controller, and a first treatment of the surgical procedure is a first distance adjacent the dentate line and a subsequent second treatment of the surgical procedure is a further distance from the dentate line.

5. The system of claim 1, wherein the tissue level indicator comprises a geometric shape and the numeric indicator is positioned within the geometric shape.

6. The system of claim 1, wherein after treatment of each treatment region within the tissue level of the axially spaced tissue levels, a region of the tissue level indicator is configured to be visually indicated as treated and regions of the tissue level not treated are not so indicated.

7. The system of claim 1, wherein a first tissue level indicator of the plurality of tissue level indicators is configured to provide an indication of a number of lesions formed and a number of lesions not formed at a first tissue level of the axially spaced tissue levels, and the first tissue level indicator remains viewable at the same time as a second tissue level of the axially spaced tissue levels is treated and a number of lesions formed and a number of lesions not formed are configured to be displayed on the second tissue level indicator of the plurality of tissue level indicators.

8. The system of claim 1, wherein each tissue level indicator of the plurality of tissue level indicators is configured to occupy a first position prior to treatment at a corresponding tissue level of the axially spaced tissue levels and a second different position during treatment at the corresponding tissue level.

9. The system of claim 1, wherein the graphic display is configured to display an electrode array icon corresponding to an electrode array of the device connected to the system.

10. The system of claim 9, wherein the graphic display is further configured to display an indication of at least one measured parameter of the electrodes of the device connected to the system.

11. A system for visually tracking distances from a fixed reference in a surgical procedure for forming a plurality of tissue lesions within a patient within a series of levels of tissue, the series of levels of tissue being axially spaced and the fixed reference determined prior to the start of application of electrosurgical energy to a first level of tissue of the series of levels of tissue, the system comprising a controller to receive an input from a clinician corresponding to a measured distance from the fixed reference to a designated region in the patient and a graphic display configured to display a series of distances computed in response to the input of the measured distance to provide a visual indication of distances from the fixed reference to the designated region for creation of lesions at different levels of tissue during the surgical procedure.

12. The system of claim 11, wherein only the distance of a selected level of tissue of the series of levels of tissue is configured to be displayed while the distances of other non-selected levels of tissue of the series of levels of tissue are not displayed.

13. The system of claim 12, wherein the measured distance is displayed simultaneously with a distance of a selected level of tissue from the fixed reference.

14. The system of claim 11, wherein when treatment of a first level of tissue of the series of levels of tissue is completed and a second level of tissue of the series of levels of tissue is selected, a first indicator corresponding to the first level of tissue and a second indicator corresponding to the second level of tissue are configured to move to different positions.

15. The system of claim 11, wherein the designated region is one of a Z-line or dentate line.

16. The system of claim 11, further comprising an adjustment button to adjust a preset value to match the measured distance.

17. The system of claim 16, wherein the adjustment button is disabled during application of electrosurgical energy.

18. A method for monitoring distances for formation of lesions at a plurality of axially spaced levels of tissue, the method comprising:
measuring a distance from a fixed reference to a designated region inside the patient; and
inputting the measured distance to a controller, wherein a graphic display is configured to provide an indication of a distance of electrodes of an electrosurgical device from the fixed reference based on an inputting of the measured distance.

19. The method of claim 18, wherein inputting the measured distance to the controller includes the step of adjusting a preset value stored by the controller to match the measured distance.

20. The method of claim 19, wherein the preset value cannot be adjusted during application of electrosurgical energy.
